(19) United States
(12) Patent Application Publication
(10) Pub. No.: US 2018/0076381 A1
(43) Pub. Date: Mar. 15, 2018

(54) METHOD FOR PRODUCING PIEZOELECTRIC ELEMENT, PIEZOELECTRIC ELEMENT, PIEZOELECTRIC DRIVE DEVICE, ROBOT, AND PUMP

(71) Applicant: Seiko Epson Corporation, Tokyo (JP)

(72) Inventors: Noboru FURUYA, Chino (JP); Hidemichi FURIHATA, Chino (JP)

(21) Appl. No.: 15/558,769

(22) PCT Filed: Feb. 9, 2016
(86) PCT No.: PCT/JP2016/000650

§ 371 Date: Sep. 15, 2017

(30) Foreign Application Priority Data
Mar. 16, 2015 (JP) 2015-052219
Mar. 16, 2015 (JP) 2015-052220
Mar. 16, 2015 (JP) 2015-052221

(51) Int. Cl. H01L 41/317; H01L 41/08; H01L 41/09; H02N 2/00; H01L 41/332; H01L 41/047; B25J 9/12; F04B 43/09

(52) U.S. Cl. CPC H01L 41/317; H01L 41/0805; H01L 41/09; H02N 2/001; A61M 5/142; H01L 41/0477; B25J 9/12; F04B 43/095; H01L 41/332

(57) ABSTRACT

A method for producing a piezoelectric element includes a step of forming a first electrode layer, a step of forming a piezoelectric body layer on the first electrode layer, a step of forming a second electrode layer on the piezoelectric body layer, a step of patterning the second electrode layer, a step of patterning the piezoelectric body layer by wet etching, and a step of forming an organic insulating layer on a side surface of the patterned piezoelectric body layer.

### BACKGROUND

### Technical Field

The present invention relates to a method for producing a piezoelectric element, a piezoelectric element, a piezoelectric drive device, a robot, and a pump.

### Background Art

A piezoelectric actuator (piezoelectric drive device) which drives a driven body by vibrating a piezoelectric body does not need a magnet or a coil, and therefore is utilized in various fields (see, for example, JP-A-2004-320979). In such a piezoelectric drive device, a piezoelectric element (bulk piezoelectric element) including a bulky piezoelectric body is generally utilized (see, for example, 2008-227123).

On the other hand, as the piezoelectric element, a piezoelectric element including a piezoelectric body in the form of a thin film (thin-film piezoelectric element) is known. The thin-film piezoelectric element is mainly utilized for performing ink injection in an inkjet printer head.

When a thin-film piezoelectric element as described above is used in a piezoelectric drive device, there is a high possibility that the piezoelectric drive device or an apparatus driven by the device can be miniaturized. In the case where a thin-film piezoelectric element is used in a piezoelectric drive device, for example, in order to prevent a short circuit between an upper electrode and a lower electrode of the piezoelectric element, or the like, it is desirable to cover a side surface of the piezoelectric body with an insulating layer. However, such an insulating layer may be peeled off in a production step after a step of forming the insulating layer, at the time of driving the piezoelectric drive device, or the like.

One object according to some embodiments of the invention is to provide a method for producing a piezoelectric element capable of suppressing peeling off of the insulating layer. Further, one object according to some embodiments of the invention is to provide a piezoelectric element capable of suppressing peeling off of the insulating layer. Further, one object according to some embodiments of the invention is to provide a piezoelectric drive device including the piezoelectric element. Further, one object according to some embodiments of the invention is to provide a robot or a pump including the piezoelectric drive device.

Further, the working accuracy of a thin-film piezoelectric element to be used in an inkjet printer head as described above or the like is high, and therefore, when such a thin-film piezoelectric element is used in a piezoelectric drive device, the cost becomes high.

One object according to some embodiments of the invention is to provide a method for producing a piezoelectric element capable of achieving cost reduction.

Further, the output of a thin-film piezoelectric element is generally significantly smaller than that of a bulk piezoelectric element. Therefore, a currently existing thin-film piezoelectric element cannot obtain a sufficient output for utilizing the element as, for example, a drive source of a motor for driving a joint of a robot in some cases.

One object according to some embodiments of the invention is to provide a piezoelectric element for an ultrasonic motor capable of achieving a high output, and a method for producing the element. Further, one object according to some embodiments of the invention is to provide an ultrasonic motor including the piezoelectric element for an ultrasonic motor. Further, one object according to some embodiments of the invention is to provide a robot or a pump including the ultrasonic motor.

### SUMMARY

The invention has been made to solve at least part of the above-mentioned problems and can be realized as the following embodiments or application examples.

### APPLICATION EXAMPLE 1

One embodiment of a method for producing a piezoelectric element according to the invention includes:

a step of forming a first electrode layer;

a step of forming a piezoelectric body layer on the first electrode layer;

a step of forming a second electrode layer on the piezoelectric body layer;

a step of patterning the second electrode layer;

a step of patterning the piezoelectric body layer by wet etching; and

a step of forming an organic insulating layer on a side surface of the patterned piezoelectric body layer.

According to such a method for producing a piezoelectric element, a side surface of the piezoelectric body layer can be formed into a concave and convex shape. According to this, the area of a contact surface between the piezoelectric body layer and the organic insulating layer can be increased. Therefore, according to such a method for producing a piezoelectric element, adhesion between the piezoelectric body layer and the organic insulating layer can be improved, and peeling off of the organic insulating layer can be suppressed.

Incidentally, in the description according to the invention, when the term ““on” is used in, for example, a sentence such as “on” a specific object (hereinafter referred to as “A”), another specific object (hereinafter referred to as “B”) is formed”, the term “on” is used while assuming that the term includes a case where B is formed directly on A, and a case where B is formed on A through another object.

### APPLICATION EXAMPLE 2

In Application Example 1, the piezoelectric body layer may be formed by repeating formation of a precursor layer by a liquid-phase method and crystallization of the precursor layer.

According to such a method for producing a piezoelectric element, a groove portion can be formed on a side surface of a piezoelectric body layer, and the side surface of the piezoelectric body layer can be formed into a concave and convex shape.

### APPLICATION EXAMPLE 3

In Application Example 1 or 2, the material of the organic insulating layer may be a photosensitive material.

According to such a method for producing a piezoelectric element, the organic insulating layer can be patterned by light exposure, development, and baking (a heat treatment) without performing etching. Therefore, according to such a method for producing a piezoelectric element, the step can be shortened, and thus, cost reduction can be achieved.

### APPLICATION EXAMPLE 4

In Application Example 3, the Young's modulus of the organic insulating layer may be 1 GPa or more.

According to such a method for producing a piezoelectric element, a force (deformation) generated in a piezoelectric body layer 40 by applying a voltage can be efficiently transmitted to the below-mentioned vibrating plate through the organic insulating layer.

### APPLICATION EXAMPLE 5

In any one of Application Examples 1 to 4, the thickness of the organic insulating layer may be 1.5 times or more and 3 times or less the thickness of the piezoelectric body layer.

According to such a method for producing a piezoelectric element, the organic insulating layer can suppress an increase in the opening area of a contact hole provided in the organic insulating layer while reliably covering the side surface of the piezoelectric body layer.

### APPLICATION EXAMPLE 6

In any one of Application Examples 1 to 5, the thickness of the piezoelectric body layer may be 1 μm or more and 10 μm or less.

According to such a method for producing a piezoelectric element, in the case where the piezoelectric element is used in an ultrasonic motor, the occurrence of a crack in the piezoelectric body layer can be suppressed while ensuring an output of the ultrasonic motor.

### APPLICATION EXAMPLE 7

One embodiment of a piezoelectric element according to the invention includes:

a first electrode layer;

a piezoelectric body layer provided on the first electrode layer;

a second electrode layer provided on the piezoelectric body layer; and

an organic insulating layer provided on a side surface of the piezoelectric body layer, wherein

the piezoelectric body layer is formed by repeating formation of a precursor layer by a liquid-phase method and crystallization of the precursor layer to form a stacked body, and patterning the stacked body by wet etching.

According to such a piezoelectric element, peeling off of the organic insulating layer can be suppressed.

### APPLICATION EXAMPLE 8

One embodiment of a piezoelectric drive device according to the invention includes:

a vibrating plate; and

the piezoelectric element according to Application Example 7 provided on a surface of the vibrating plate.

According to such a piezoelectric drive device, the device includes the piezoelectric element according to the invention, and therefore, has high reliability.

### APPLICATION EXAMPLE 9

One embodiment of a robot according to the invention includes:

a plurality of link portions;

a joint portion for connecting the plurality of link portions; and

the piezoelectric drive device according to Application Example 8 which rotates the plurality of link portions at the joint portion.

According to such a robot, the robot can include the piezoelectric drive device according to the invention.

### APPLICATION EXAMPLE 10

One embodiment of a pump according to the invention includes:

the piezoelectric drive device according to Application Example 8;

a tube for transporting a liquid; and

a plurality of fingers for blocking the tube by driving the piezoelectric drive device.

According to such a pump, the pump can include the piezoelectric drive device according to the invention.

### APPLICATION EXAMPLE 11

One embodiment of a method for producing a piezoelectric element according to the invention includes:

a step of forming a first electrode layer;

a step of forming a piezoelectric body layer on the first electrode layer;

a step of forming a second electrode layer on the piezoelectric body layer;

a step of forming a resist layer on the second electrode layer;

a step of patterning the second electrode layer by wet etching;

a step of patterning the piezoelectric body layer by wet etching; and

a step of removing an eaves portion of the second electrode layer generated by side etching in the step of patterning the piezoelectric body layer by wet etching.

According to such a method for producing a piezoelectric element, the piezoelectric body layer and the second electrode layer are patterned by wet etching. Therefore, according to such a method for producing a piezoelectric element, as compared with the case where the piezoelectric body layer and the second electrode layer are patterned by dry etching, cost reduction can be achieved. For example, when the piezoelectric body layer or the second electrode layer of 1 μm is etched, it takes about 10 minutes in the case of dry etching, but etching can be achieved in about 2 minutes in the case of wet etching. Further, in the case of wet etching, a resist layer used as a mask for etching can be easily peeled off with a solution of acetone or the like, and peeling off of the resist layer and cleaning of a wafer (a substrate with the piezoelectric body layer and the like formed thereon) can be performed simultaneously. On the other hand, in the case of dry etching, the resist layer is denatured, and therefore, necessity to perform asking or the like occurs, and the resist layer cannot be peeled off by a simple step. Further, the price of an etching device for wet etching is lower than the price of an etching device for dry etching. Therefore, according to the method for producing a piezoelectric element in which the piezoelectric body layer and the second electrode layer are patterned by wet etching, cost reduction can be achieved.

### APPLICATION EXAMPLE 12

In Application Example 11,

the step of forming the second electrode layer may include
a step of forming an adhesion layer, and
a step of forming an electrically conductive layer on the adhesion layer, and

in the step of removing the eaves portion,
after the adhesion layer is removed, the electrically conductive layer may be removed.

According to such a method for producing a piezoelectric element, the electrically conductive layer in the eaves portion can be removed in a short time. For example, when the electrically conductive layer is tried to be removed before removing the adhesion layer, an area coming into contact with an etching liquid is small, and it takes time to remove the electrically conductive layer in some cases.

### APPLICATION EXAMPLE 13

In Application Example 11 or 12, the second electrode layer may contain at least one of copper and gold.

According to such a method for producing a piezoelectric element, the resistance of the second electrode layer can be decreased as compared with the second electrode layer composed of, for example, iridium.

### APPLICATION EXAMPLE 14

In any one of Application Examples 11 to 13, the thickness of the second electrode layer may be 50 nm or more and 10 μm or less.

According to such a method for producing a piezoelectric element, an increase in the size of the piezoelectric element can be suppressed while decreasing the resistance of the second electrode layer.

### APPLICATION EXAMPLE 15

In any one of Application Examples 1 to 14, the thickness of the piezoelectric body layer may be 1 μm or more and 10 μm or less.

According to such a method for producing a piezoelectric element, in the case where the piezoelectric element is used in an ultrasonic motor, the occurrence of a crack in the piezoelectric body layer can be suppressed while ensuring an output of the ultrasonic motor.

### APPLICATION EXAMPLE 16

One embodiment of a piezoelectric element for an ultrasonic motor according to the invention includes:

a first electrode layer;

a piezoelectric body layer provided on the first electrode layer; and

a second electrode layer provided on the piezoelectric body layer, wherein

the second electrode layer contains copper, and

the thickness of the second electrode layer is 50 nm or more and 10 μm or less.

According to such a piezoelectric element for an ultrasonic motor, an increase in the size of the piezoelectric element can be suppressed while decreasing the resistance of the second electrode layer. According to such a piezoelectric element for an ultrasonic motor, by decreasing the resistance of the second electrode layer, in the case where the element is used in an ultrasonic motor, a high output can be achieved.

### APPLICATION EXAMPLE 17

In Application Example 16, the second electrode layer may include

an adhesion layer,

an electrically conductive layer provided on the adhesion layer and containing the copper, and

an antioxidation layer provided on the electrically conductive layer.

According to such a piezoelectric element for an ultrasonic motor, oxidation of the electrically conductive layer can be prevented by the antioxidation layer.

### APPLICATION EXAMPLE 18

In Application Example 16 or 17, the material of the antioxidation layer may be the same as the material of the adhesion layer.

According to such a piezoelectric element for an ultrasonic motor, the antioxidation layer can be formed using the same sputtering device as the sputtering device used for forming the adhesion layer (using the same sputtering target), and therefore, cost reduction can be achieved.

### APPLICATION EXAMPLE 19

In Application Example 16 or 17, the material of the antioxidation layer may be a polymer.

According to such a piezoelectric element for an ultrasonic motor, the antioxidation layer can be formed by dipping the electrically conductive layer in, for example, a chemical liquid containing a polymer, and the antioxidation layer can be formed by a simple method.

### APPLICATION EXAMPLE 20

One embodiment of a method for producing a piezoelectric element for an ultrasonic motor according to the invention includes:

a step of forming a first electrode layer;

a step of forming a piezoelectric body layer on the first electrode layer; and

a step of forming a second electrode layer on the piezoelectric body layer, wherein

the second electrode layer contains copper, and

the thickness of the second electrode layer is 50 nm or more and 10 μm or less.

According to such a method for producing a piezoelectric element for an ultrasonic motor, an increase in the size of the piezoelectric element can be suppressed while decreasing the resistance of the second electrode layer. According to such a piezoelectric element for an ultrasonic motor, by decreasing the resistance of the second electrode layer, in the case where the element is used in an ultrasonic motor, a high output can be achieved.

### APPLICATION EXAMPLE 21

In Application Example 20,

the step of forming the second electrode layer may include
a step of forming an adhesion layer,
a step of forming an electrically conductive layer containing the copper on the adhesion layer, and
a step of forming an antioxidation layer on the electrically conductive layer.

According to such a method for producing a piezoelectric element for an ultrasonic motor, oxidation of the electrically conductive layer can be prevented by the antioxidation layer.

### APPLICATION EXAMPLE 22

In Application Example 20 or 21, the material of the adhesion layer and the material of the antioxidation layer may be the same.

According to such a method for producing a piezoelectric element for an ultrasonic motor, the antioxidation layer can be formed using the same sputtering device as the sputtering device used for forming the adhesion layer, and therefore, cost reduction can be achieved.

### APPLICATION EXAMPLE 23

In Application Example 20 or 21, the material of the antioxidation layer may be a polymer.

According to such a piezoelectric element for an ultrasonic motor, the antioxidation layer can be formed by dipping the electrically conductive layer in, for example, a chemical liquid containing a polymer, and the antioxidation layer can be formed by a simple method.

### APPLICATION EXAMPLE 24

One embodiment of an ultrasonic motor according to the invention includes:

a vibrating plate; and

the piezoelectric element for an ultrasonic motor according to any one of Application Examples 16 to 19 provided on a surface of the vibrating plate.

According to such an ultrasonic motor, the ultrasonic motor includes the piezoelectric element for an ultrasonic motor according to the invention, and therefore, a high output can be achieved.

### APPLICATION EXAMPLE 25

One embodiment of a robot according to the invention includes:

a plurality of link portions;

a joint portion for connecting the plurality of link portions; and

the ultrasonic motor according to Application Example 24 which rotates the plurality of link portions at the joint portion.

According to such a robot, the robot can include the ultrasonic motor according to the invention.

### APPLICATION EXAMPLE 26

One embodiment of a pump according to the invention includes:

the ultrasonic motor according to Application Example 24;

a tube for transporting a liquid; and

a plurality of fingers for blocking the tube by driving the ultrasonic motor.

According to such a pump, the pump can include the ultrasonic motor according to the invention.

### BRIEF DESCRIPTION OF DRAWINGS

FIG. 1 is a cross-sectional view schematically showing a piezoelectric element according to this embodiment.

FIG. 2 is a flowchart for illustrating a method for producing a piezoelectric element according to this embodiment.

FIG. 3 is a cross-sectional view schematically showing a step of producing a piezoelectric element according to this embodiment.

FIG. 4 is a cross-sectional view schematically showing a step of producing a piezoelectric element according to this embodiment.

FIG. 5 is a cross-sectional view schematically showing a step of producing a piezoelectric element according to this embodiment.

FIG. 6 is a cross-sectional view schematically showing a step of producing a piezoelectric element according to this embodiment.

FIG. 7 is a cross-sectional view schematically showing a step of producing a piezoelectric element according to this embodiment.

FIG. 8 is a cross-sectional view schematically showing a step of producing a piezoelectric element according to this embodiment.

FIG. 9 is a cross-sectional view schematically showing a step of producing a piezoelectric element according to this embodiment.

FIG. 10 is a cross-sectional view schematically showing a step of producing a piezoelectric element according to this embodiment.

FIG. 11 is a cross-sectional view schematically showing a step of producing a piezoelectric element according to this embodiment.

FIG. 12 is a cross-sectional view schematically showing a step of producing a piezoelectric element according to this embodiment.

FIG. 13 is a cross-sectional view schematically showing a step of producing a piezoelectric element according to this embodiment.

FIG. 14 is a cross-sectional view schematically showing a piezoelectric element according to this embodiment.

FIG. 15A is a result of SEM observation.

FIG. 15B is a result of SEM observation.

FIG. 15C is a result of SEM observation.

FIG. 16A is a graph showing the sheet resistance of each material.

FIG. 16B is a graph showing the sheet resistance of each material.

FIG. 17 is a cross-sectional view schematically showing a piezoelectric element according to a variation of this embodiment.

FIG. 18 is a plan view schematically showing a piezoelectric element according to a variation of this embodiment.

FIG. 19A is a plan view schematically showing a piezoelectric drive device according to this embodiment.

FIG. 19B is a cross-sectional view schematically showing a piezoelectric drive device according to this embodiment.

FIG. 20 is a plan view schematically showing a vibrating plate of a piezoelectric drive device according to this embodiment.

FIG. 21 is a view for illustrating an electrical connection state between a piezoelectric drive device according to this embodiment and a drive circuit.

FIG. 22 is a view for illustrating an operation of a piezoelectric drive device according to this embodiment.

FIG. 23 is a view for illustrating a robot according to this embodiment.

FIG. 24 is a view for illustrating a wrist portion of a robot according to this embodiment.

FIG. 25 is a view for illustrating a pump according to this embodiment.

### DETAILED DESCRIPTION

Hereinafter, preferred embodiments of the invention will be described in detail with reference to the drawings. Note that the embodiments described below are not intended to unduly limit the content of the invention described in the claims. Further, not all the configurations described below are necessarily essential components of the invention.

### 1. Piezoelectric Element

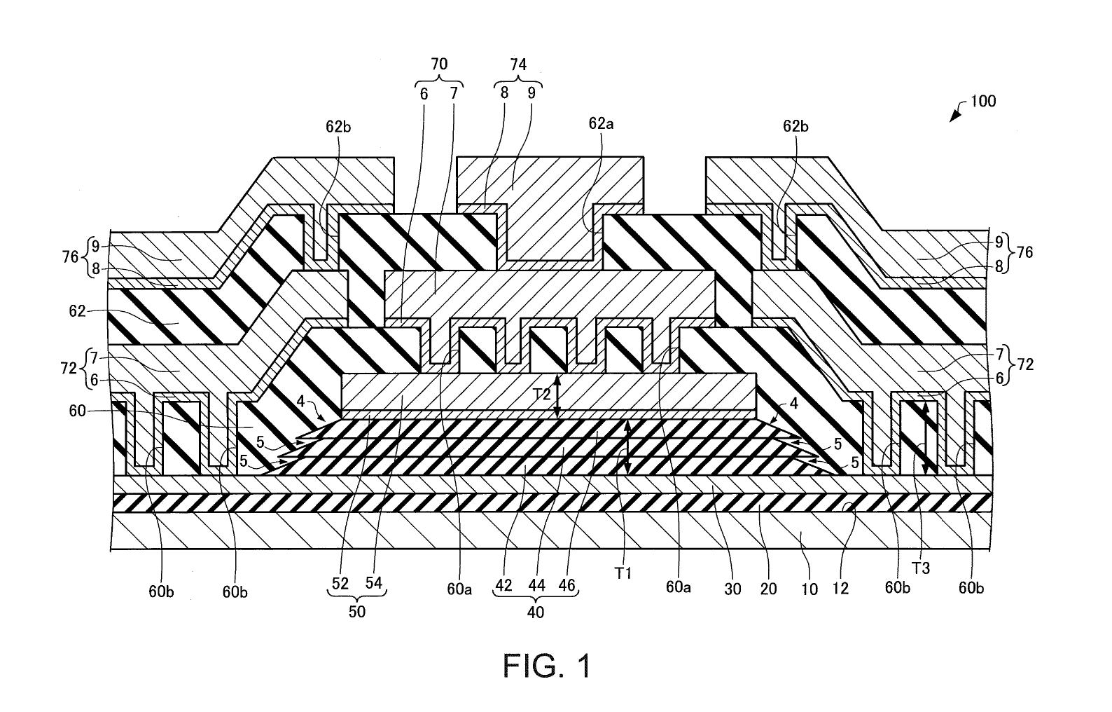

First, a piezoelectric element according to this embodiment will be described with reference to the drawings. FIG. 1 is a cross-sectional view schematically showing a piezoelectric element 100 according to this embodiment.

As shown in FIG. 1, the piezoelectric element 100 includes a substrate 10, a foundation layer 20, a first electrode layer 30, a piezoelectric body layer 40, a second electrode layer 50, organic insulating layers 60 and 62, and wiring layers 70, 72, 74, and 76.

The shape of the substrate 10 is a flat plate shape. The substrate 10 is, for example, a semiconductor substrate (specifically, a silicon substrate). The substrate 10 can be deformed according to the deformation of the piezoelectric body layer 40.

The foundation layer 20 is provided on the substrate 10. The foundation layer 20 may be constituted by an oxide silicon layer provided on the substrate 10 and a zirconium oxide layer provided on the silicon oxide layer. The foundation layer 20 can function as an etching stopper layer when etching the first organic insulating layer 60. The foundation layer 20 can be deformed according to the deformation of the piezoelectric body layer 40.

The first electrode layer 30 is provided on the foundation layer 20. The first electrode layer 30 may be constituted by an iridium layer provided on the foundation layer 20 and a platinum layer provided on the iridium layer. The thickness of the iridium layer is, for example, 5 nm or more and 100 nm or less, preferably about 20 nm. The thickness of the platinum layer is, for example, 50 nm or more and 300 nm or less, preferably about 130 nm. The first electrode layer 30 is one electrode for applying a voltage to the piezoelectric body layer 40. Incidentally, the material of the first electrode layer 30 may be only one type of metal material such as Ti, Pt, Ta, Ir, Sr, In, Sn, Au, Al, Fe, Cr, Ni, or Cu, or a mixed material or a stacked material of two or more types of these metal materials.

The piezoelectric body layer 40 is provided on the first electrode layer 30. The piezoelectric body layer 40 is constituted by, for example, a plurality of layers. In the example shown in the drawing, the piezoelectric body layer 40 is constituted by a first layer 42 provided on the first electrode layer 30, a second layer 44 provided on the first layer 42, and a third layer 46 provided on the second layer 44.

Incidentally, for convenience sake, in FIG. 1, the piezoelectric body layer 40 composed of three layers 42, 44, and 46 is shown, however, the number of layers constituting the piezoelectric body layer 40 is not particularly limited, and is appropriately determined according to the thickness T1 of the piezoelectric body layer 40. For example, in the case of the piezoelectric body layer 40 having a thickness of 1 μm, the piezoelectric body layer 40 may be constituted by 5 to 6 layers.

The width of the lower surface of the first layer 42 of the piezoelectric body layer 40 is larger than the width of the lower surface of the second layer 44. The width of the lower surface of the second layer 44 is larger than the width of the lower surface of the third layer 46. In the example shown in the drawing, the widths of the layers 42, 44, and 46 decrease toward the second electrode layer 50 side from the first electrode layer 30 side. The side surface of each of the layers 42, 44, and 46 is inclined with respect to the upper surface 12 of the substrate 10. In the example shown in the drawing, the angles of inclination with respect to the upper surface 12 of the side surfaces of the respective layers 42, 44, and 46 are the same.

On the side surface 4 of the piezoelectric body layer 40, a groove portion 5 is provided. The groove portion 5 is constituted by the end portion of each of the layers 42, 44, and 46. A plurality of groove portions 5 are provided according to the number of layers constituting the piezoelectric body layer 40. It can also be said that the side surface 4 of the piezoelectric body layer 40 has a concave and convex shape due to the end portions of the layers 42, 44, and 46.

The thickness T1 of the piezoelectric body layer 40 is, for example, 1 μm or more and 10 μm or less, preferably 1.5 μm or more and 7 μm or less, more preferably about 3 μm. When the thickness of the piezoelectric body layer 40 is less than 1 μm, in the case where the piezoelectric body layer 40 is used in an ultrasonic motor, the output of the ultrasonic motor may be insufficient in some cases. Specifically, when the application voltage to the piezoelectric body layer 40 is increased for trying to increase the output, the piezoelectric body layer 40 may cause electrical breakdown in some cases. When the thickness of the piezoelectric body layer 40 is 1 μm, a voltage of 20 V to 40 V can be applied to the piezoelectric body layer 40. When the thickness of the piezoelectric body layer 40 is more than 10 μm, a crack may occur in the piezoelectric body layer 40 in some cases.

As the piezoelectric body layer 40, a perovskite-type oxide piezoelectric material is used. Specifically, the material of the piezoelectric body layer 40 is lead zirconate titanate (Pb(Zr,Ti)O₃:PZT) or lead zirconate titanate niobate (Pb(Zr,Ti,Nb)O₃:PZTN).

The second electrode layer 50 is provided on the piezoelectric body layer 40. The thickness T2 of the second electrode layer 50 is, for example, 50 nm or more and 10 μm or less, preferably 1 μm or more and 7 μm or less, more preferably about 1.0 μm. When the thickness of the second electrode layer 50 is less than 50 nm, the resistance of the second electrode layer 50 may be high in some cases. For example, the resistance of the entire piezoelectric element 100 is in a saturated state when the thickness of the second electrode layer 50 is 10 μm, and even if the thickness of the second electrode layer 50 is increased to more than 10 μm, the resistance of the entire piezoelectric element 100 cannot be decreased, but the thickness of the second electrode layer 50 becomes large. The second electrode layer 50 is the other electrode for applying a voltage to the piezoelectric body layer 40. In the example shown in the drawing, the second electrode layer 50 includes an adhesion layer 52 provided on the piezoelectric body layer 40 and an electrically conductive layer 54 provided on the adhesion layer 52.

The thickness of the adhesion layer 52 of the second electrode layer 50 is, for example, 10 nm or more and 100 nm or less, preferably about 50 nm. The adhesion layer 52 is, for example, a TiW layer, a Ti layer, a Cr layer, an NiCr layer, or a stacked body thereof. The adhesion layer 52 can improve the adhesion property between the piezoelectric body layer 40 and the electrically conductive layer 54. Incidentally, in the case where the material of the piezoelectric body layer 40 is PZT, the adhesion layer 52 is preferably a TiW layer. According to this, the suppression of deformation of the piezoelectric body layer 40 can be prevented by the adhesion layer 52.

The thickness of the electrically conductive layer 54 of the second electrode layer 50 is, for example, 1 μm or more and 10 μm or less. When the thickness of the electrically conductive layer 54 is less than 1 μm, the resistance of the second electrode layer 50 may be high in some cases. When the thickness of the electrically conductive layer 54 is more than 10 μm, the size of the piezoelectric element 100 may be large in some cases. The electrically conductive layer 54 is, for example, a Cu layer, an Au layer, an Al layer, or a stacked body thereof. That is, the electrically conductive layer 54 contains at least one of copper and gold. By the electrically conductive layer 54, the resistance of the second electrode layer 50 can be decreased.

The first organic insulating layer 60 is provided on the side surface 4 of the piezoelectric body layer 40. Specifically, the first organic insulating layer 60 is provided so as to cover the side surface 4 of the piezoelectric body layer 40. The groove portion 5 is filled with the first organic insulating layer 60. In the example shown in the drawing, the first organic insulating layer 60 is also provided on the electrode layers 30 and 50. The thickness T3 of the first organic insulating layer 60 (the thickness of the first organic insulating layer 60 located on the first electrode layer 30) is, for example, 1.5 times or more and 3 times or less the thickness T1 of the piezoelectric body layer 40. When the thickness of the first organic insulating layer 60 is smaller than 1.5 times the thickness of the piezoelectric body layer 40, the side surface 4 of the piezoelectric body layer 40 cannot be covered therewith in some cases. When the thickness of the first organic insulating layer 60 is larger than 3 times the thickness of the piezoelectric body layer 40, the opening areas of contact holes 60a and 60b provided in the first organic insulating layer 60 may be large in some cases. Specifically, the thickness of the first organic insulating layer 60 is 1.5 μm or more and 30 μm or less, preferably 2 μm or more and 10 μm or less, more preferably about 3 μm.

The material of the first organic insulating layer 60 is an organic material. Specifically, the material of the first organic insulating layer 60 is an epoxy-based resin, an acrylic resin, a polyimide-based resin, a silicone-based resin, or the like. The material of the first organic insulating layer 60 is a photosensitive material. The “photosensitive” refers to a property that a substance causes a chemical reaction by light. Specifically, the first organic insulating layer 60 can be patterned by light exposure, development, and baking (a heat treatment) without using etching. The Young's modulus of the first organic insulating layer 60 is, for example, 1 GPa or more. The Young's modulus of the first organic insulating layer 60 may be determined based on JIS K7161. The heat resistance of the first organic insulating layer 60 is preferably high, and the deflection temperature under load (thermal deformation temperature) of the first organic insulating layer 60 is preferably, for example, 200° C. or higher.

The first wiring layer 70 is connected to the second electrode layer 50. The first wiring layer 70 is provided in the first contact hole 60a provided on the second electrode layer 50 of the first organic insulating layer 60. A plurality of first contact holes 60a are provided, and the number of first contact holes is not particularly limited. The first wiring layer 70 is provided on the first organic insulating layer 60.

The second wiring layer 72 is connected to the first electrode layer 30. The second wiring layer 72 is provided in the second contact hole 60b provided on the first electrode layer 30 of the first organic insulating layer 60. A plurality of second contact holes 60b are provided, and the number of second contact holes is not particularly limited. The second wiring layer 72 is provided on the first organic insulating layer 60. The second wiring layer 72 is provided so as to sandwich the piezoelectric body layer 40 (on both lateral sides of the piezoelectric body layer 40).

The first wiring layer 70 and the second wiring layer 72 each include, for example, a seed layer 6 and an electrically conductive layer 7 provided on the seed layer 6. The thickness of the seed layer 6 is, for example, 50 nm or more and 100 nm or less. The seed layer 6 is, for example, a TiW layer, a Ti layer, a Cr layer, an NiCr layer, or a stacked body thereof. In particular, when considering electric corrosion (electrochemical corrosion), the seed layer 6 is preferably a TiW layer. The thickness of the electrically conductive layer 7 is, for example, 1 μm or more and 10 μm or less. The electrically conductive layer 7 is, for example, a Cu layer, an Ni layer, an Au layer, an Al layer, or a stacked body thereof.

The second organic insulating layer 62 is provided on the first organic insulating layer 60 so as to cover the wiring layers 70 and 72. The thickness and the material of the second organic insulating layer 62 may be the same as the thickness and the material of the first organic insulating layer 60.

The third wiring layer 74 is connected to the first wiring layer 70. The third wiring layer 74 is provided in a third contact hole 62a provided on the first wiring layer 70 of the second organic insulating layer 62. The third wiring layer 74 is further provided on the second organic insulating layer 62.

The fourth wiring layer 76 is connected to the second wiring layer 72. The fourth wiring layer 76 is provided in a fourth contact hole 62b provided on the second wiring layer 72 of the second organic insulating layer 62. The fourth wiring layer 76 is further provided on the second organic insulating layer 62.

The third wiring layer 74 and the fourth wiring layer 76 each include, for example, a seed layer 8 and an electrically conductive layer 9 provided on the seed layer 8. The thickness and the material of the seed layer 8 may be the same as the thickness and the material of the seed layer 6. The thickness of the electrically conductive layer 9 is, for example, 1 μm or more and 10 μm or less. The electrically conductive layer 9 is, for example, a stacked body obtained by stacking a Cu layer, an Ni layer, and an Au layer in this order, and the thickness of the Ni layer is about 2 μm, and the thickness of the Au layer is 300 nm or less. By the Ni layer, a reaction between the Cu layer and the Au layer can be suppressed. Further, by the Au layer, when bonding to a wiring of the below-mentioned ultrasonic motor, the wiring and the wiring layers 74 and 76 can be bonded by the Au layers (gold-gold bonding).

Incidentally, in the above description, an example in which two organic insulating layers are provided is described, however, the number of organic insulating layers is not particularly limited. In addition, also the number of wiring layers is not particularly limited.

### 2. Method for Producing Piezoelectric Element

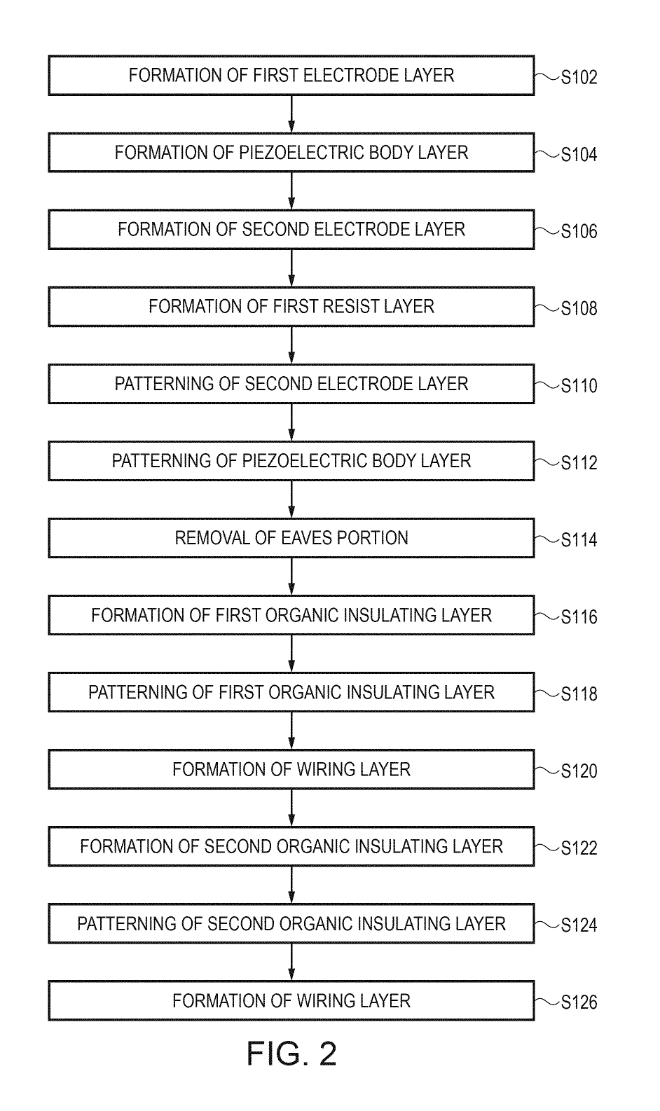

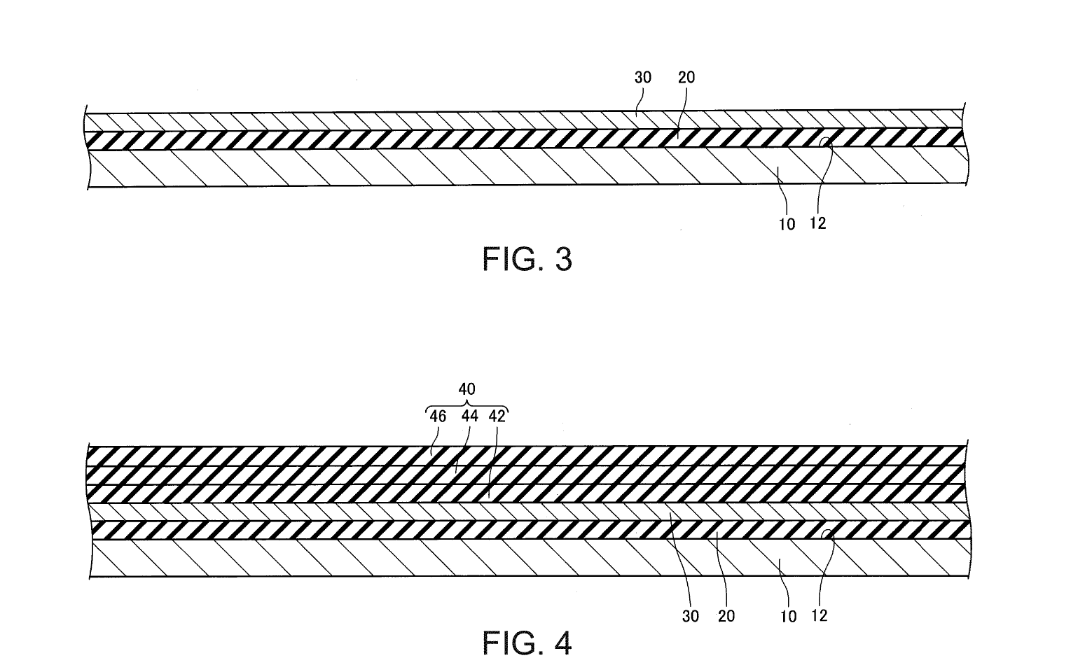

Next, a method for producing the piezoelectric element 100 according to this embodiment will be described with reference to the drawings. FIG. 2 is a flowchart for illustrating the method for producing the piezoelectric element 100 according to this embodiment. FIGS. 3 to 13 are cross-sectional views schematically showing steps of producing the piezoelectric element 100 according to this embodiment.

As shown in FIG. 3, the foundation layer 20 is formed on the substrate 10, and the first electrode layer 30 is formed on the foundation layer 20 (S102). Specifically, after a silicon oxide layer is formed by thermally oxidizing the substrate (silicon substrate) 10, a zirconium layer is formed on the silicon oxide layer, and then, a zirconium oxide layer is formed by thermally oxidizing the zirconium layer, whereby the foundation layer 20 composed of the silicon oxide layer and the zirconium oxide layer is formed. The zirconium layer is formed by, for example, a sputtering method or a CVD method (Chemical Vapor Deposition). The first electrode layer 30 is formed by, for example, a sputtering method, a CVD method, or a vacuum deposition method.

As shown in FIG. 4, on the first electrode layer 30, the piezoelectric body layer (stacked body) 40 is formed (S104). The piezoelectric body layer 40 is formed by, for example, repeating formation of a precursor layer by a liquid-phase method and crystallization of the precursor layer. In the example shown in the drawing, on the first electrode layer 30, a first precursor layer is formed, and the first precursor layer is crystallized, whereby the first layer 42 is formed. Subsequently, on the layer of the first layer 42, a second precursor layer is formed, and the second precursor layer is crystallized, whereby the second layer 44 is formed. Subsequently, on the layer of the second layer 44, a third precursor layer is formed, and the third precursor layer is crystallized, whereby the third layer 46 is formed. One precursor layer is formed by, for example, repeating application by a liquid-phase method and drying (degreasing) three times. The crystallization is performed by, for example, firing at 600° C. or higher and 1200° C. or lower.

Incidentally, the liquid-phase method is a method of depositing a thin film material using a raw material liquid containing a constituent material of a thin film (piezoelectric body layer), and specifically, a sol-gel method, an MOD (Metal Organic Deposition) method, or the like.

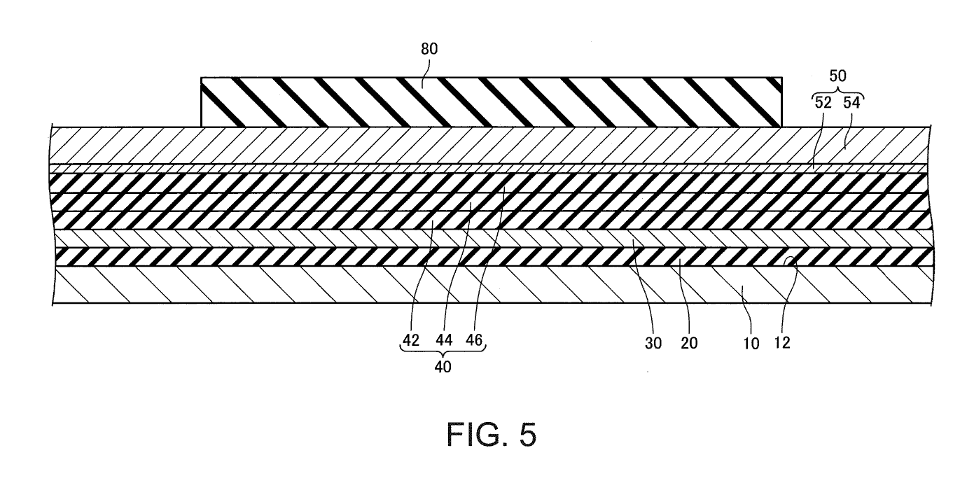

As shown in FIG. 5, the second electrode layer 50 is formed on the piezoelectric body layer 40 (S106). Specifically, this step includes a step of forming the adhesion layer 52 and a step of forming the electrically conductive layer 54 on the adhesion layer 52. The adhesion layer 52 and the electrically conductive layer 54 are formed by, for example, a sputtering method, a CVD method, a vacuum deposition method, or a plating method. Subsequently, on the second electrode layer 50, a first resist layer 80 having a predetermined shape is formed (S108). The first resist layer 80 is formed by, for example, photolithography.

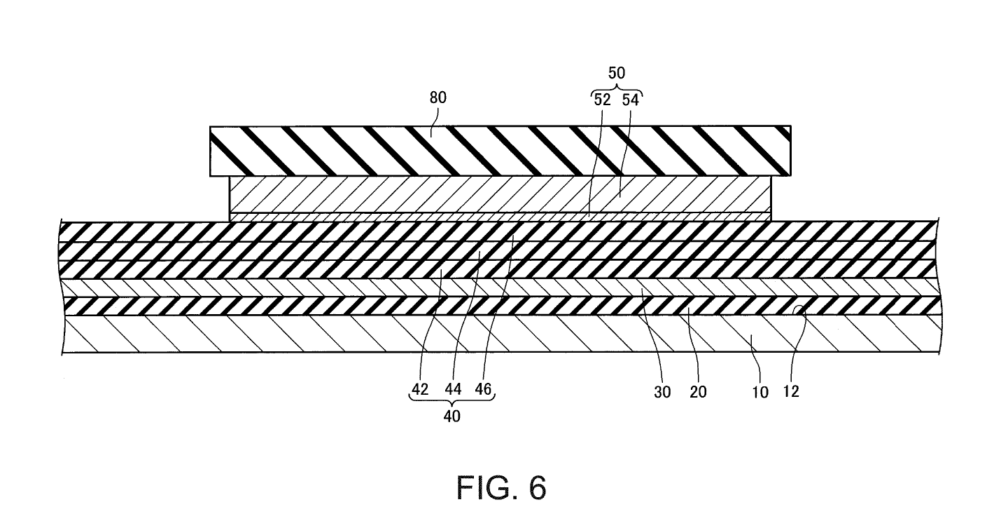

As shown in FIG. 6, the second electrode layer 50 is patterned by wet etching using the first resist layer 80 as a mask (S110). Specifically, first, the electrically conductive layer 54 of the second electrode layer 50 is etched, and subsequently, the adhesion layer 52 of the second electrode layer 50 is etched. As an etching liquid for the etching of the adhesion layer 52, for example, in the case where the adhesion layer 52 is a TiW layer, an aqueous hydrogen peroxide solution is used. As an etching liquid for the etching of the electrically conductive layer 54, for example, in the case where the electrically conductive layer 54 is a Cu layer, ammonium persulfate is used.

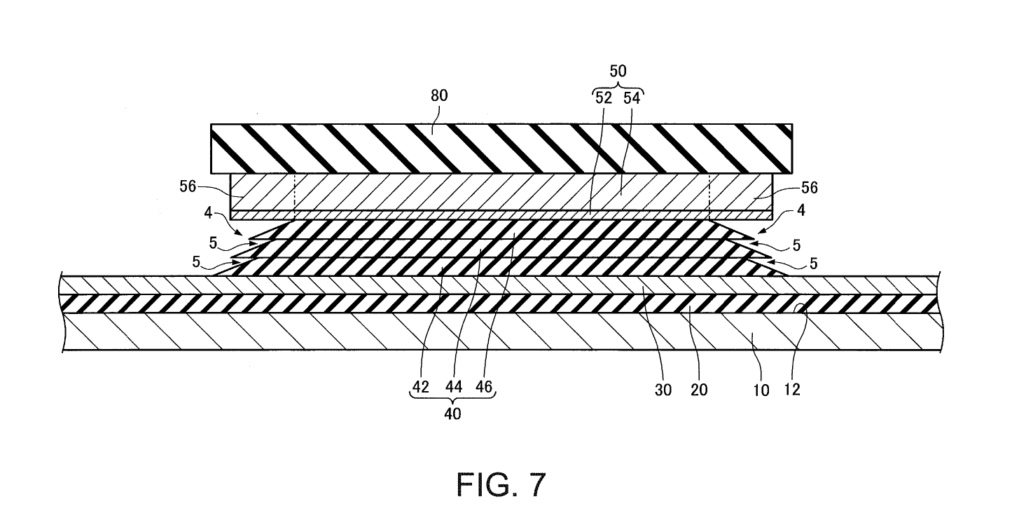

As shown in FIG. 7, the piezoelectric body layer 40 is patterned by wet etching using the second electrode layer 50 as a mask (S112). As an etching liquid, for example, in the case where the material of the piezoelectric body layer 40 is PZT, a mixed liquid containing at least one or more of hydrochloric acid, nitric acid, and hydrofluoric acid is used. In this step, on the side surface 4 of the piezoelectric body layer 40, the groove portion 5 is formed. Here, in the above-mentioned firing for crystallization of the precursor layer, lead in each precursor layer has a distribution in the thickness direction, and the number of lead elements is increased on the upper side. In an etching liquid in this step, as the number of lead elements is larger, the etching speed is faster, and therefore, as shown in FIG. 7, the layers 42, 44, and 46 of the piezoelectric body layer 40 have a tapered shape in which the width becomes narrower upward, and the groove portions 5 are formed on the side surface 4 of the piezoelectric body layer 40. Further, in this step, the piezoelectric body layer 40 is side-etched, and the second electrode layer 50 has an eaves portion 56. The eaves portion 56 is a portion of the second electrode layer 50 which is not in contact with the upper surface of the piezoelectric body layer 40, and is a portion located above the side surface 4 of the piezoelectric body layer 40 in the example shown in the drawing.

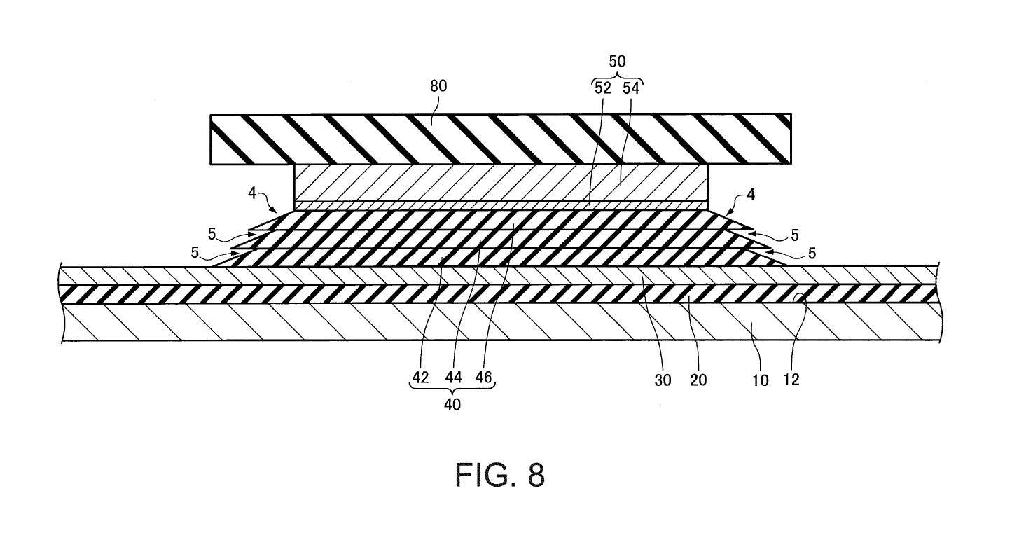

As shown in FIG. 8, the eaves portion 56 of the second electrode layer 50 generated by side etching in the step of patterning the piezoelectric body layer 40 (S112) is removed by wet etching (S114). Specifically, first, the adhesion layer 52 of the eaves portion 56 is removed, and subsequently, the electrically conductive layer 54 of the eaves portion 56 is removed. As an etching liquid for the etching of the adhesion layer 52, for example, the etching liquid used in the step of patterning the second electrode layer 50 (S110) is used. Thereafter, for example, by using acetone or the like as a peeling liquid, the first resist layer 80 is removed. Incidentally, after this step, the first electrode layer 30 may be patterned in a desired shape.

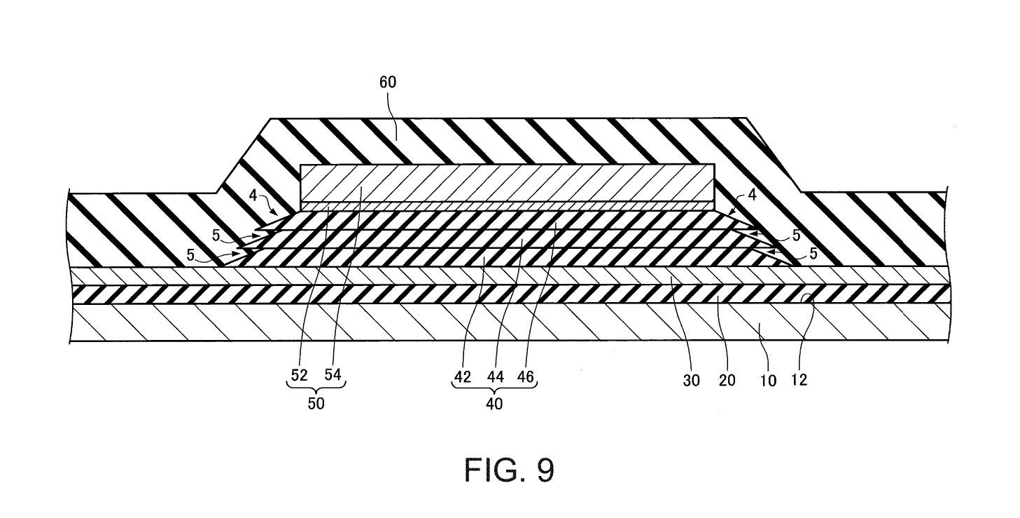

As shown in FIG. 9, on the side surface 4 of the patterned piezoelectric body layer 40, the first organic insulating layer is formed (S116). Specifically, the first organic insulating layer 60 is formed so as to cover the side surface 4 of the piezoelectric body layer 40, the upper surface of the first electrode layer 30, and the upper surface and the side surface of second electrode layer 50. The first organic insulating layer 60 is formed by, for example, a spin coating method or a CVD method.

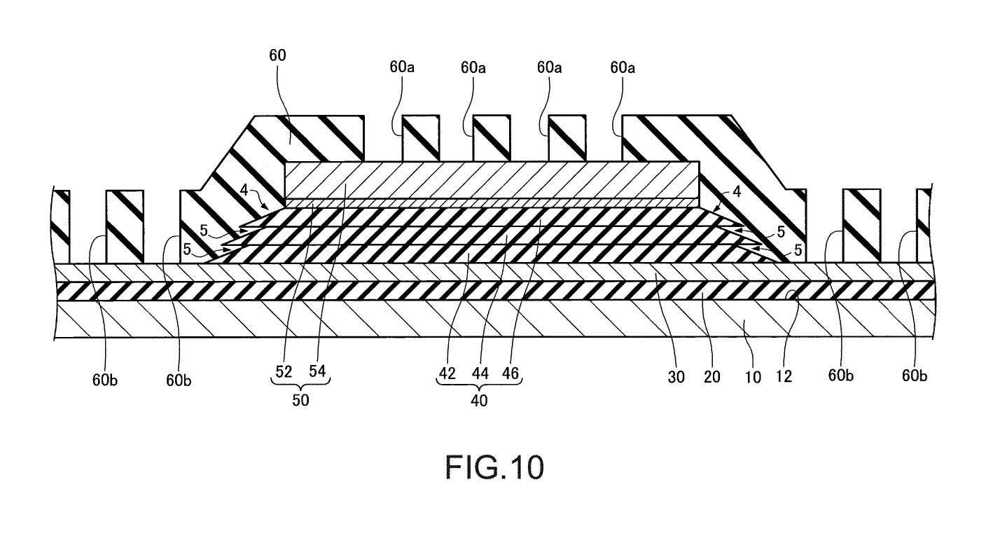

As shown in FIG. 10, the first organic insulating layer 60 is patterned, whereby the contact holes 60a and 60b are formed (S118). In the case where the material of the first organic insulating layer 60 is a photosensitive material, the first organic insulating layer 60 can be patterned by light exposure, development, and baking without performing etching. Incidentally, in the case where the material of the first organic insulating layer 60 is not a photosensitive material, the first organic insulating layer 60 is patterned by photolithography and etching.

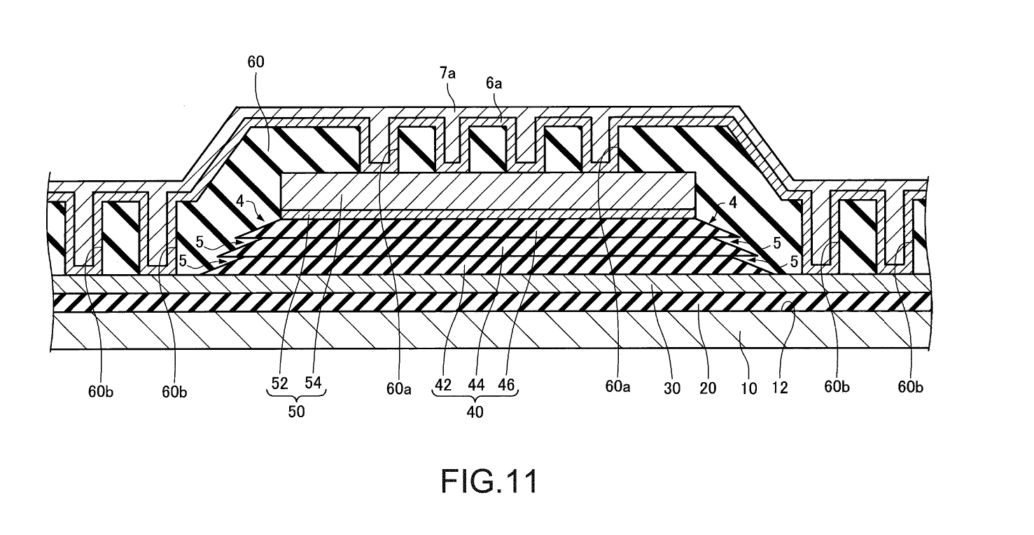

As shown in FIG. 11, on the first organic insulating layer 60, and in the contact holes 60a and 60b, a seed layer 6a is formed, and on the seed layer 6a, a first electrically conductive layer 7a is formed. The seed layer 6a and the first electrically conductive layer 7a are formed by, for example, a sputtering method or a CVD method. The thickness of the first electrically conductive layer 7a is, for example, 100 nm or more and 500 nm or less.

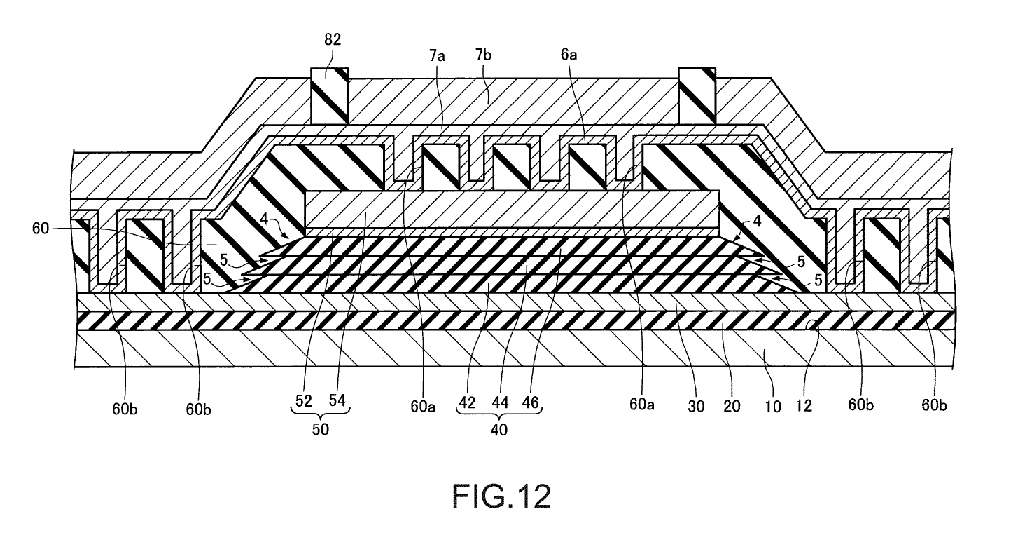

As shown in FIG. 12, on the first electrically conductive layer 7a, a second resist layer 82 having a predetermined shape is formed. The second resist layer 82 is formed by, for example, photolithography. Subsequently, by a plating method (electroplating method), a second electrically conductive layer 7b is grown on the first electrically conductive layer 7a. Thereafter, the second resist layer 82 is removed. The second resist layer 82 is removed by the same method as used for the first resist layer 80.

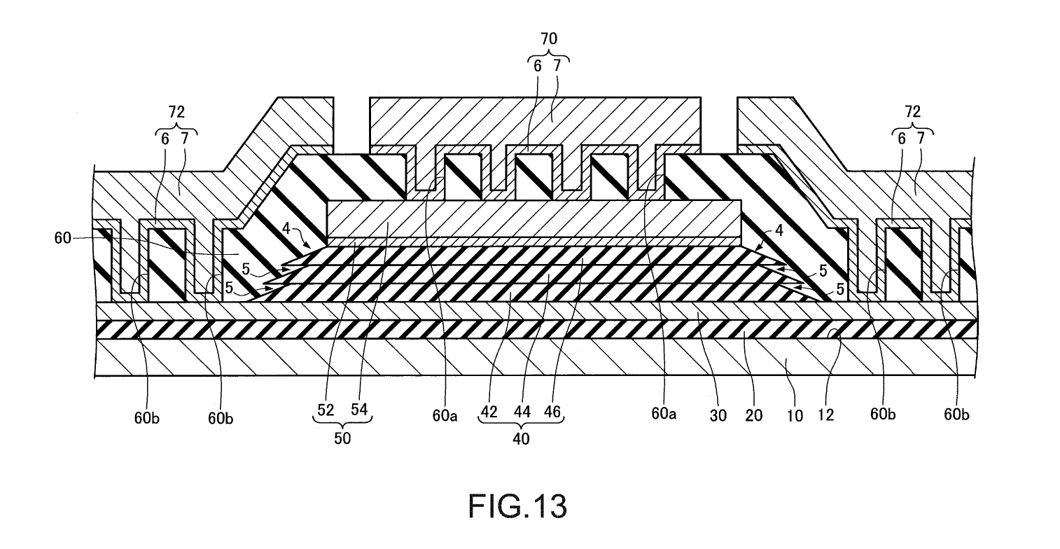

As shown in FIG. 13, the entire surface (the seed layer 6a and the electrically conductive layers 7a and 7b) is wet-etched to expose a part of the first organic insulating layer 60, whereby the seed layer 6 composed of the seed layer 6a and the electrically conductive layer 7 composed of the electrically conductive layers 7a and 7b are formed. As described above, the wiring layers 70 and 72 can be formed by a so-called semi-additive method (S120).

As shown in FIG. 1, on the wiring layers 70 and 72, the second organic insulating layer 62 is formed (S122), and the second organic insulating layer 62 is patterned, whereby the contact holes 62a and 62b are formed (S124). The second organic insulating layer 62 is formed by, for example, the same method as used for the first organic insulating layer 60, and patterned by the same method as used for the first organic insulating layer 60. Subsequently, on the second organic insulating layer 62 and in the contact holes 62a and 62b, the wiring layers 74 and 76 are formed (S126). The wiring layers 74 and 76 are formed by the same method as used for the wiring layers 70 and 72. Incidentally, in the case where the wiring layers 74 and 76 include an Ni layer and an Au layer on the Cu layer, the Ni layer and the Au layer may be formed by an electroless plating method.

By the above-mentioned steps, the piezoelectric element 100 can be produced.

The piezoelectric element 100 and the method for producing the same have, for example, the following characteristics.

In the method for producing the piezoelectric element 100, the piezoelectric body layer 40 is patterned by wet etching, and on the side surface 4 of the patterned piezoelectric body layer 40, the first organic insulating layer 60 is formed. Therefore, on the side surface 4 of the piezoelectric body layer 40, for example, the groove portion 5 can be formed, and thus, the side surface 4 can be formed into a concave and convex shape. Due to this, the area of the contact surface between the piezoelectric body layer 40 and the first organic insulating layer 60 can be increased. Therefore, according to the method for producing the piezoelectric element 100, the adhesion property between the piezoelectric body layer 40 and the first organic insulating layer 60 can be improved, and peeling off of the first organic insulating layer 60 can be suppressed.

In the method for producing the piezoelectric element 100, the piezoelectric body layer 40 is formed by repeating formation of a precursor layer by a liquid-phase method and crystallization of the precursor layer. Therefore, in the method for producing the piezoelectric element 100, the groove portion 5 can be formed on the side surface 4 of the patterned piezoelectric body layer 40, and thus, the side surface 4 can be formed into a concave and convex shape.

In the method for producing the piezoelectric element 100, the material of the organic insulating layers 60 and 62 is a photosensitive material. Therefore, the organic insulating layers can be patterned by light exposure, development, and baking without performing etching. Therefore, in the method for producing the piezoelectric element 100, the step can be shortened, and thus, cost reduction can be achieved.

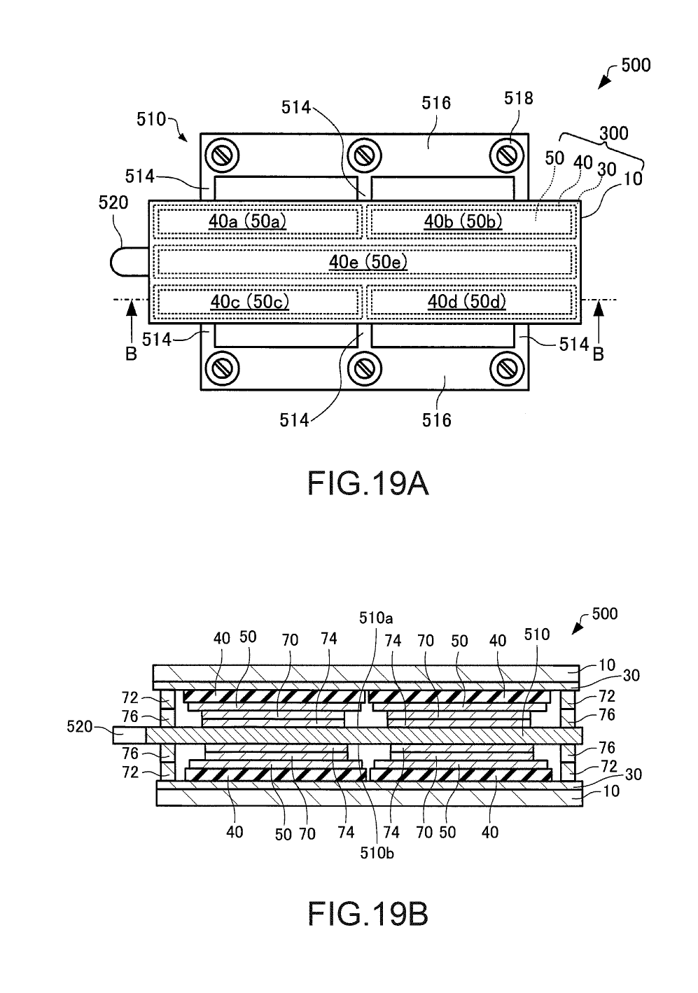

In the method for producing the piezoelectric element 100, the Young's modulus of the organic insulating layers 60 and 62 is 1 GPa or more. Therefore, a force (deformation) generated in the piezoelectric body layer 40 by applying a voltage can be efficiently transmitted to the below-mentioned vibrating plate 510 (see FIG. 19) through the organic insulating layers 60 and 62. For example, when the Young's modulus of the organic insulating layers 60 and 62 is less than 1 GPa, the organic insulating layers 60 and 62 absorb the force generated in the piezoelectric body layer 40, and the force transmitted to the vibrating plate may be decreased in some cases.

In the method for producing the piezoelectric element 100, the thickness T3 of the first organic insulating layer 60 is 1.5 times or more and 3 times or less the thickness T1 of the piezoelectric body layer 40. Therefore, in the method for producing the piezoelectric element 100, the first organic insulating layer 60 can suppress an increase in the opening areas of the contact holes 60a and 60b while reliably covering the side surface 4 of the piezoelectric body layer 40.

In the method for producing the piezoelectric element 100, the thickness T1 of the piezoelectric body layer 40 is 1 μm or more and 10 μm or less. According to this, in the case where the piezoelectric element 100 is used in an ultrasonic motor, the occurrence of a crack in the piezoelectric body layer 40 can be suppressed while ensuring an output of the ultrasonic motor.

In the method for producing the piezoelectric element 100, the piezoelectric body layer 40 and the second electrode layer 50 are patterned by wet etching. Therefore, in the method for producing the piezoelectric element 100, as compared with the case where the piezoelectric body layer and the second electrode layer are patterned by dry etching, cost reduction can be achieved. For example, when the piezoelectric body layer or the second electrode layer of 1 μm is etched, it takes about 10 minutes in the case of dry etching, but etching can be achieved in about 2 minutes in the case of wet etching. Further, in the case of wet etching, a resist layer used as a mask for etching can be easily peeled off with a solution of acetone or the like, and peeling off of the resist layer and cleaning of a wafer (a substrate with the piezoelectric body layer and the like formed thereon) can be performed simultaneously. On the other hand, in the case of dry etching, the resist layer is denatured, and therefore, necessity to perform asking or the like occurs, and the resist layer cannot be peeled off by a simple step. Further, the price of an etching device for wet etching is lower than the price of an etching device for dry etching. Therefore, in the method for producing the piezoelectric element 100 in which the piezoelectric body layer and the second electrode layer are patterned by wet etching, cost reduction can be achieved. Further, when a layer composed of gold or copper is etched by dry etching, the inside of an etching device may be contaminated in some cases. Further, when a piezoelectric body layer is etched by dry etching, etching damage to the first electrode layer may be caused in some cases. In the method for producing the piezoelectric element 100, such a problem of device contamination or etching damage can be avoided.

In the method for producing the piezoelectric element 100, the eaves portion 56 is removed by wet etching. Therefore, a short circuit between the first electrode layer 30 and the second electrode layer 50 can be prevented. For example, if the eaves portion 56 remains, the eaves portion 56 may break through the first organic insulating layer 60 to cause a short circuit between the first electrode layer 30 and the second electrode layer 50 in some cases.

In the method for producing the piezoelectric element 100, in the step of removing the eaves portion 56 (S114), after the adhesion layer 52 is removed, the electrically conductive layer 54 is removed. Therefore, the electrically conductive layer 54 of the eaves portion 56 can be removed in a short time. For example, when the electrically conductive layer is tried to be removed before removing the adhesion layer, an area of the electrically conductive layer coming into contact with an etching liquid is small, and therefore, it may take time to remove the electrically conductive layer in some cases.

In the method for producing the piezoelectric element 100, the thickness T2 of the second electrode layer 50 is 50 nm or more and 10 μm or less. According to this, an increase in the size of the piezoelectric element 100 can be suppressed while decreasing the resistance of the second electrode layer 50. By decreasing the resistance of the second electrode layer 50, the efficiency of the applied voltage can be improved, and further, the amount of heat generated by the resistance of the second electrode layer 50 can be reduced. Further, a thin-film piezoelectric element has a larger capacitance than a bulk piezoelectric element, and therefore, the impedance of the piezoelectric body layer is decreased. Therefore, by decreasing the resistance of the second electrode layer 50, the impedance of the piezoelectric body layer can be increased, and a voltage to be applied to the piezoelectric body layer can be increased. As a result, in the case where the piezoelectric element 100 is used in an ultrasonic motor, a high output can be achieved.

In the method for producing the piezoelectric element 100, the second electrode layer 50 contains at least one of copper and gold. Therefore, the resistance of the second electrode layer 50 can be decreased as compared with the second electrode layer 50 composed of, for example, iridium. Incidentally, copper has a higher binding property (is more likely to bind to another material) than gold, and therefore has a high adhesion property to the first organic insulating layer 60. Due to this, the outermost surface of the second electrode layer 50 is preferably copper.

In the piezoelectric element 100, the second electrode layer 50 contains copper, and the thickness T2 of the second electrode layer 50 is 50 nm or more and 10 μm or less. According to this, an increase in the size of the piezoelectric element 100 can be suppressed while decreasing the resistance of the second electrode layer 50. By decreasing the resistance of the second electrode layer 50, the efficiency of the applied voltage can be improved, and further, the amount of heat generated by the resistance of the second electrode layer 50 can be reduced. Further, a thin-film piezoelectric element has a larger capacitance than a bulk piezoelectric element, and therefore, the impedance of the piezoelectric body layer is decreased. Therefore, by decreasing the resistance of the second electrode layer 50, the impedance of the piezoelectric body layer can be increased, and the voltage to be applied to the piezoelectric body layer can be increased. As a result, in the case where the piezoelectric element 100 is used in an ultrasonic motor, a high output can be achieved.

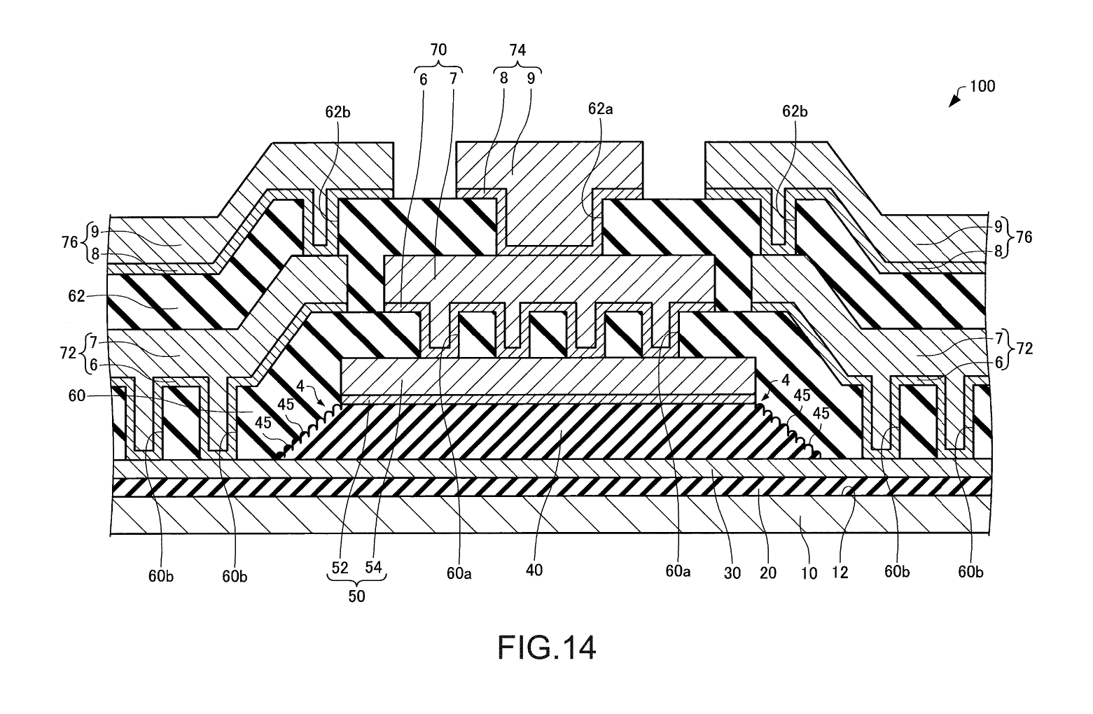

Incidentally, in the above description, an example in which the piezoelectric body layer 40 is formed by a liquid-phase method is described, however, the method for forming the piezoelectric body layer 40 is not particularly limited, and may be a PVD (Physical Vapor Deposition) method such as a sputtering or a laser abrasion method. For example, when the piezoelectric body layer 40 is formed by a sputtering method, on the side surface 4 formed by wet etching, a plurality of convex portions 45 having an upward convex domed shape are formed as shown in FIG. 14. This is because when the piezoelectric body layer 40 is formed by a sputtering method, the piezoelectric body layer 40 has a columnar crystal structure. In the case where the piezoelectric body layer 40 is formed by a sputtering method, as shown in FIG. 14, the piezoelectric body layer 40 may be formed by forming a precursor layer having a predetermined thickness at a time and crystallizing the precursor layer without repeating formation of a precursor layer and crystallization of the precursor layer.

Further, in the above-mentioned example, the wiring layers 70, 72, 74, and 76 are formed by a so-called semi-additive method, however, the wiring layers 70, 72, 74, and 76 may be formed by a so-called subtractive method. That is, the wiring layers 70, 72, 74, and 76 may be formed by forming a seed layer and an electrically conductive layer by a sputtering method or the like, forming a resist layer on the electrically conductive layer, and etching the electrically conductive layer and the seed layer using the resist layer as a mask.

### 3. Experimental Examples

Hereinafter, the invention will be more specifically described by showing experimental examples. Incidentally, the invention is by no means limited to the following experimental examples.

### 3.1. SEM Observation

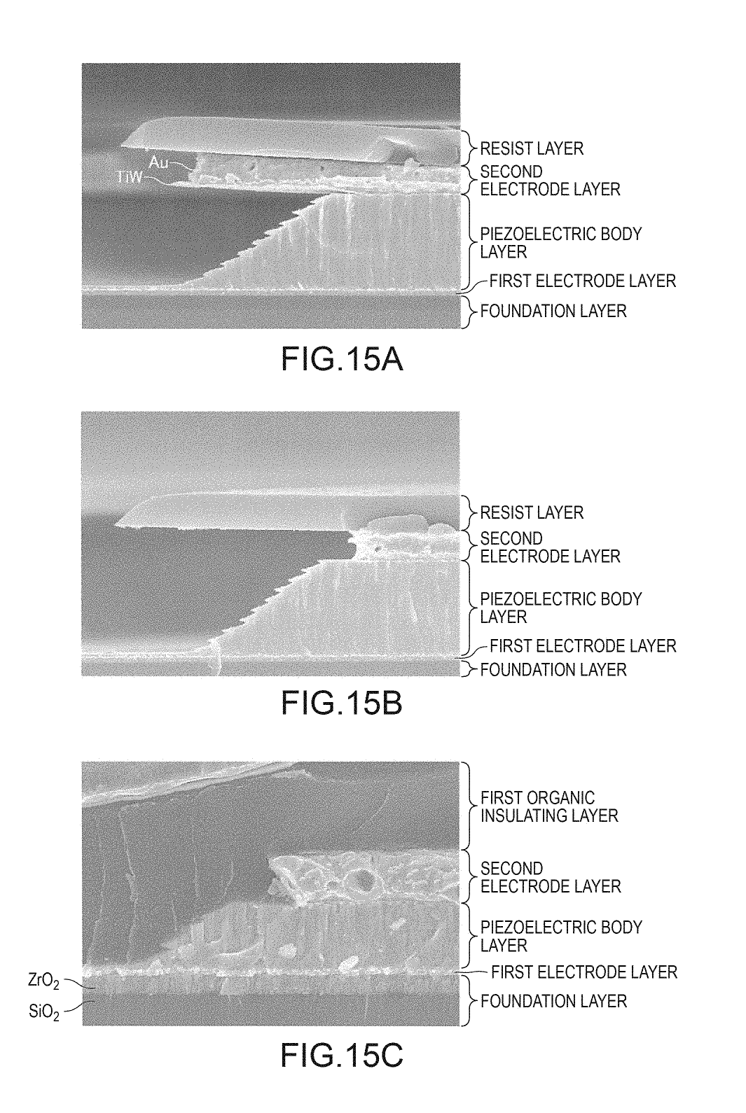

FIGS. 15A, 15B, and 15C are SEM photographs of cross sections in the steps of producing the piezoelectric element according to the experimental examples. FIG. 15A is a photograph after the step of patterning the piezoelectric body layer 40 (S112), FIG. 15B is a photograph after the step of removing the eaves portion 56 (S114), and FIG. 15C is a photograph after completion of all steps. As the foundation layer, a stacked body of an SiO₂ layer and a ZrO₂ layer was used. As the first electrode, a Pt layer was used. As the piezoelectric body layer, a PZT layer was used. As the second electrode layer, a stacked body of a TiW layer and an Au layer was used. As the organic insulating layer, an acrylic photosensitive insulating film was used. The TiW layer was wet-etched using an aqueous hydrogen peroxide solution. The Au layer was wet-etched using an iodine-based mixed solvent.

From FIGS. 15A, 15B, and 15C, it was found that groove portions are formed on the side surface of the piezoelectric body layer, and the surface has a concave and convex shape. It was also found that an eaves portion is generated in the second electrode layer by side etching in the step of patterning the piezoelectric body layer, and the eaves portion can be removed by wet etching.

### 3.2. Measurement of Sheet Resistance

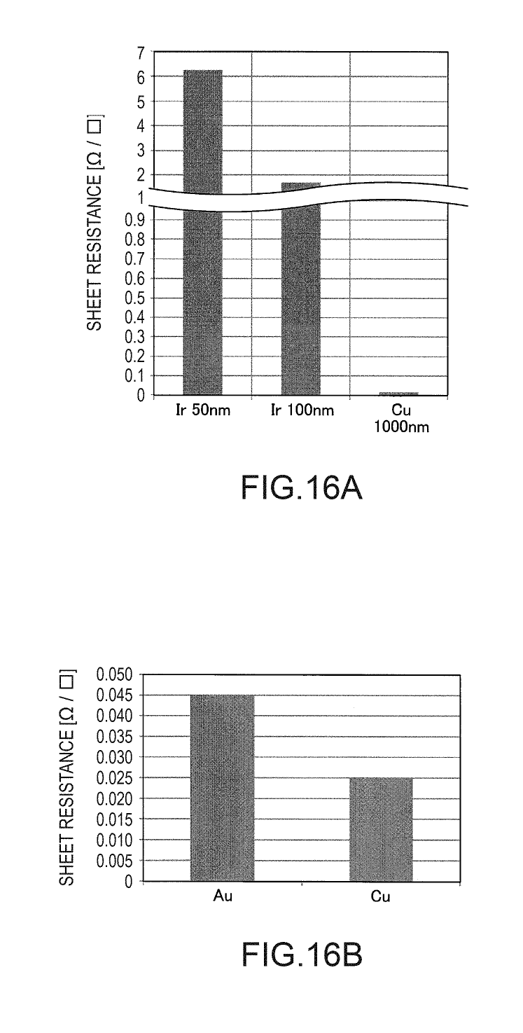

FIGS. 16A and 16B are graphs showing the sheet resistance of each material. In FIG. 16A, the sheet resistances of an Ir layer (50 nm), an Ir layer (100 nm), and a Cu layer (1000 nm) are shown. In FIG. 16B, the sheet resistances of an Au layer (1 μm) and a Cu layer (1 μm) are shown. From FIGS. 16A and 16B, it is found that copper has a lower sheet resistance than iridium and gold.

### 4. Variations of Piezoelectric Element

### 4.1. First Variation

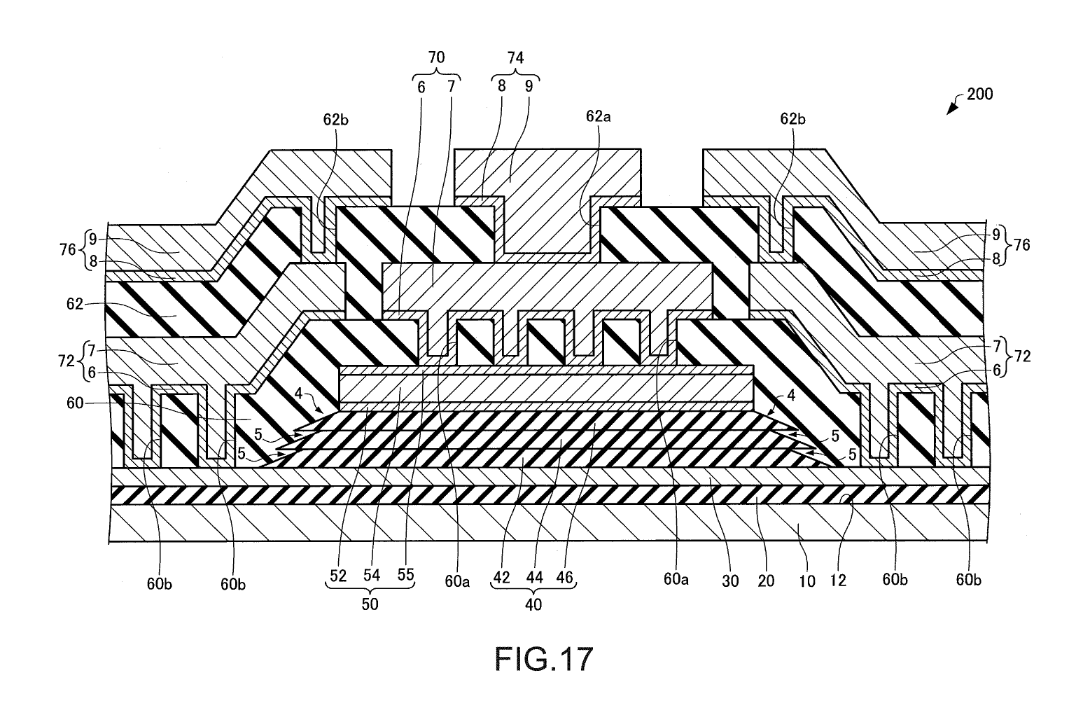

Next, a piezoelectric element according to a first variation of this embodiment will be described with reference to the drawing. FIG. 17 is a cross-sectional view schematically showing a piezoelectric element 200 according to the first variation of this embodiment.

Hereinafter, with respect to the piezoelectric element 200 according to the first variation of this embodiment, members having the same function as the constituent members of the piezoelectric element 100 according to this embodiment are denoted by the same reference numerals, and a detailed description thereof is omitted. This also applies to a piezoelectric element according to a second variation of this embodiment described later.

As shown in FIG. 17, the piezoelectric element 200 is different from the above-mentioned piezoelectric element 100 in that the second electrode layer 50 includes an antioxidation layer 55 provided on the electrically conductive layer 54. The antioxidation layer 55 can prevent oxidation of the electrically conductive layer 54.

The antioxidation layer 55 is, for example, a TiW layer, a Ti layer, a Cr layer, an NiCr layer, or a stacked body thereof. The material of the antioxidation layer 55 may be the same as the material of the adhesion layer 52. The antioxidation layer 55 is formed by, for example, a sputtering method or a CVD method. By using the same material as the material of the antioxidation layer 52 for the material of the adhesion layer 55, the antioxidation layer 55 can be formed using, for example, the same sputtering device as the sputtering device used for forming the adhesion layer 52 (using the same sputtering target), and therefore, cost reduction can be achieved.

The material of the antioxidation layer 55 may be a polymer. Specifically, the material of the antioxidation layer 55 may be a thiazole-based or imidazole-based mixed polymer. The thickness of the antioxidation layer 55 composed of a polymer is, for example, several nanometers or less. The antioxidation layer 55 composed of a polymer is formed by, for example, dipping the electrically conductive layer 54 in a chemical liquid containing a polymer. In this manner, the antioxidation layer 55 composed of a polymer can be formed by a simple method. A treatment for forming the antioxidation layer 55 composed of a polymer is performed after forming the electrically conductive layer 54, and further, may also be performed after removing the eaves portion 56 and removing the first resist layer 80. In addition, a treatment for forming the antioxidation layer composed of a polymer may be performed after forming the electrically conductive layer 7 of the wiring layers 70 and 72, and the electrically conductive layer 9 of the wiring layers 74 and 76. That is, the wiring layers 70 and 72 may include the antioxidation layer provided on the electrically conductive layer 7. Further, the wiring layers 74 and 76 may include the antioxidation layer provided on the electrically conductive layer 9. According to this, oxidation of the electrically conductive layers 7 and 9 can be prevented.

### 4.2. Second Variation

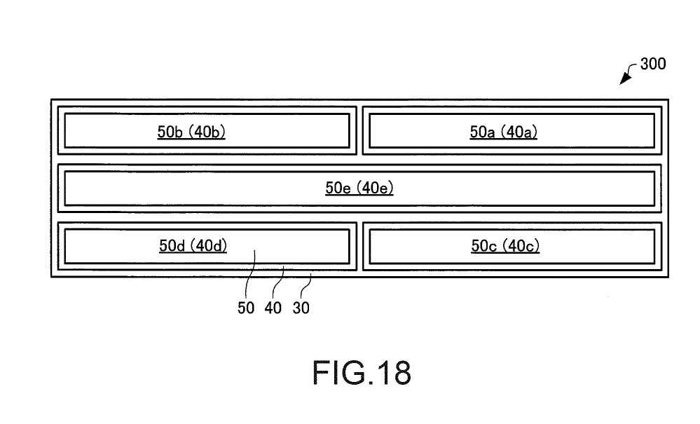

Next, a piezoelectric element according to a second variation of this embodiment will be described with reference to the drawing. FIG. 18 is a plan view schematically showing a piezoelectric element 300 according to the second variation of this embodiment. Incidentally, for convenience sake, in FIG. 18, illustration of the organic insulating layers 60 and 62 and the wiring layers 70, 72, 74, and 76 is omitted.

In the above-mentioned piezoelectric element 100, one piezoelectric body layer 40 is included as shown in FIG. 1. On the other hand, in the piezoelectric element 300, a plurality of piezoelectric body layers 40 are included as shown in FIG. 18.

In the piezoelectric element 300, the first electrode layer 30 is used as a common electrode, and a plurality of piezoelectric body layers 40 are provided on the first electrode layer 30. The number of piezoelectric body layers 40 is not particularly limited, however, in the example shown in the drawing, five piezoelectric body layers 40 are provided. The five piezoelectric body layers 40a, 40b, 40c, 40d, and 40e are separated from each other. In the example shown in the drawing, the areas of the piezoelectric body layers 40a, 40b, 40c, and 40d are the same, and the piezoelectric body layer 40e has a larger area than the piezoelectric body layers 40a, 40b, 40c, and 40d. The piezoelectric body layers 40a and 40b are provided side by side in the longitudinal direction of the piezoelectric body layers, the piezoelectric body layers 40c and 40d are provided side by side in the longitudinal direction of the piezoelectric body layers, and the piezoelectric body layer 40e is provided between the piezoelectric body layers 40a and 40b and the piezoelectric body layers 40c and 40d. The planar shape of each piezoelectric body layer 40 is, for example, a rectangle.

A plurality of second electrode layers 50 are provided according to the number of piezoelectric body layers 40. In the example shown in the drawing, five second electrode layers 50 are provided, and the second electrode layers 50a, 50b, 50c, 50d, and 50e are provided on the piezoelectric body layers 40a, 40b, 40c, 40d, and 40e, respectively. The planar shape of each second electrode layer 50 is, for example, a rectangle.

Incidentally, the first electrode layer 30 may not be one common electrode, but five first electrode layers 30 having the same planar shape as the second electrode layers 50 may be provided. Further, the piezoelectric body layers 40a, 40b, 40c, 40d, and 40e may not be separated from each other and may be one continuous piezoelectric body layer.

### 5. Piezoelectric Drive Device

Next, a piezoelectric drive device (ultrasonic motor) 500 according to this embodiment will be described with reference to the drawings. FIG. 19A is a plan view schematically showing the piezoelectric drive device 500 according to this embodiment. FIG. 19B is a cross-sectional view taken along the line B-B of FIG. 19A schematically showing the piezoelectric drive device 500 according to this embodiment. The piezoelectric drive device 500 includes the piezoelectric element according to the invention. Hereinafter, the piezoelectric drive device 500 including the above-mentioned piezoelectric element 300 as the piezoelectric element according to the invention will be described. Incidentally, for convenience sake, in FIGS. 19A and 19B, the piezoelectric element 300 is shown in a simplified manner.

As shown in FIGS. 19A and 19B, the piezoelectric drive device 500 includes the piezoelectric element 300 and a vibrating plate 510. The piezoelectric drive device 500 includes the piezoelectric element 300, and therefore can have high reliability.

Two piezoelectric elements 300 are provided interposing the vibrating plate 510 therebetween. The two piezoelectric elements 300 may be provided symmetrically with respect to the vibrating plate 510. In the example shown in the drawing, the piezoelectric elements 300 are provided on a first surface 510a and a second surface 510b of the vibrating plate 510. The piezoelectric elements 300 are provided so that the wiring layers 74 and 76 face toward the vibrating plate 510. Although not shown in the drawings, on the first surface 510a and the second surface 510b, a gold wiring is provided, and the piezoelectric elements 300 may be provided on the vibrating plate 510 by gold-gold bonding between the gold wiring and the gold layer of the wiring layers 74 and 76. Incidentally, the piezoelectric elements 300 may be adhered to the vibrating plate 510 with an electrically conductive adhesive.

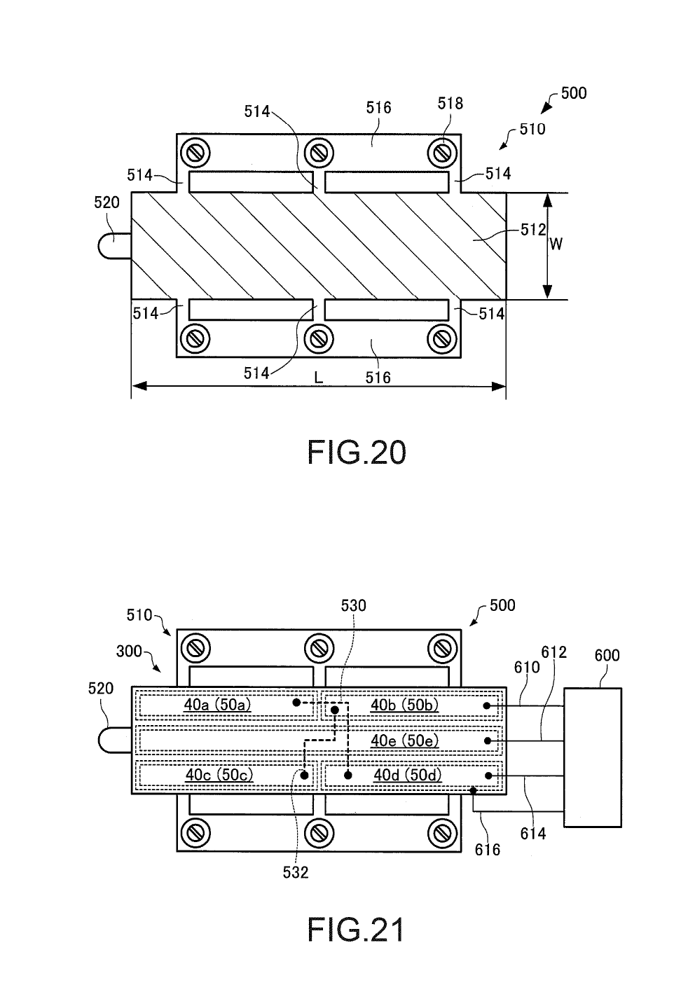

The vibrating plate 510 is provided between the two piezoelectric elements 300. Here, FIG. 20 is a plan view schematically showing the vibrating plate 510. As shown in FIG. 20, the vibrating plate 510 includes a rectangular vibrating body portion 512, connecting portions 514, three of which extend from each of the right and left long sides of the vibrating body portion 512, and two attaching portions 516 connected to the three connecting portions 514 on the right and left sides, respectively. Incidentally, for convenience sake, in FIG. 20, the vibrating body portion 512 is hatched. The attaching portions 516 are used for attaching the piezoelectric drive device 500 to another member with a screw 518. The material of the vibrating plate 510 is, for example, a metal material such as a stainless steel, aluminum, an aluminum alloy, titanium, a titanium alloy, copper, a copper alloy, or an iron-nickel alloy, a ceramic material such as alumina or zirconia, silicon, or the like.

On the upper surface (first surface 510a) and the lower surface (second surface 510b) of the vibrating body portion 512, the piezoelectric element 100 is provided. The ratio of the length L to the width W of the vibrating body portion 512 is preferably set as follows: L:W=about 7:2. This ratio is a preferred value for the vibrating body portion 512 to perform ultrasonic vibrations (described later) such that it bends right and left along its plane. The length L of the vibrating body portion 512 is, for example, 3.5 mm or more and 30 mm or less, and the width W thereof is, for example, 1 mm or more and 8 mm or less. Incidentally, in order for the vibrating body portion 512 to perform ultrasonic vibrations, the length L is preferably, 50 mm or less. The thickness of the vibrating body portion 512 (the thickness of the vibrating plate 510) is, for example, 50 μm or more and 700 μm or less. When the thickness of the vibrating body portion 512 is 50 μm or more, the vibrating body portion has sufficient rigidity for supporting the piezoelectric element 300. Further, when the thickness of the vibrating body portion 512 is 700 μm or less, a sufficiently large deformation can be caused in response to deformation of the piezoelectric element 100.

On one short side of the vibrating plate 510, a protrusion portion 520 (also referred to as “contact portion” or “operation portion”) is provided. The protrusion portion 520 is a member for applying a force to a driven body by coming into contact with the driven body. The protrusion portion 520 is preferably formed from a material having durability such as a ceramic (for example, Al₂O₃).

FIG. 21 is a view for illustrating an electrical connection state between the piezoelectric drive device 500 and a drive circuit 600. Incidentally, for convenience sake, in FIG. 21, the piezoelectric element 300 is shown in a simplified manner. Among the five second electrode layers 50a, 50b, 50c, 50d, and 50e, a pair of second electrode layers 50a and 50d disposed at diagonal positions are electrically connected to each other through a wiring 530, a pair of second electrode layers 50b and 50c disposed at the other diagonal positions are electrically connected to each other through a wiring 532. The wirings 530 and 532 may be formed by a film formation treatment, or may be realized by a wire-shaped wiring. The three second electrode layers 50b, 50e, and 50d disposed on the right side in FIG. 21 and the first electrode layer 30 are electrically connected to the drive circuit 600 through wirings 610, 612, 614, and 616, respectively.

The drive circuit 600 can rotate a rotor (driven body) coming into contact with the protrusion portion 520 in a predetermined rotation direction by applying a cyclically varying AC voltage or pulsating voltage between the pair of second electrode layers 50a and 50d and the first electrode layer 30 to cause the piezoelectric drive device 500 to perform ultrasonic vibrations. Here, the “pulsating voltage” refers to a voltage obtained by adding a DC offset to the AC voltage, and the direction of the voltage (electric field) is one direction from one electrode toward the other electrode. Further, the drive circuit 600 can rotate the rotor coming into contact with the protrusion portion 520 in the opposite direction by applying an AC voltage or a pulsating voltage between the other pair of second electrode layers 50b and 50c and the first electrode layer 30. The application of such a voltage is performed simultaneously in the two piezoelectric elements 300 provided on both surfaces of the vibrating plate 510. In the example shown in FIG. 21, the piezoelectric body layers 40a and 40d are driven simultaneously. Further, the piezoelectric body layers 40b and 40c are driven simultaneously. Incidentally, for convenience sake, in FIG. 19, illustration of the wirings 530, 532, 610, 612, 614, and 616 is omitted.

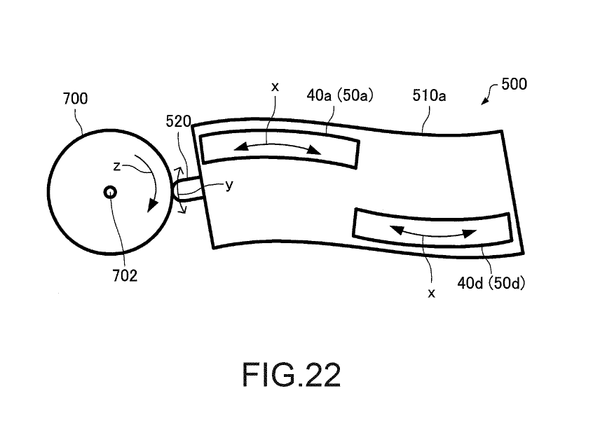

FIG. 22 is a view for illustrating an operation of the piezoelectric drive device 500 according to this embodiment. As shown in FIG. 22, the protrusion portion 520 of the piezoelectric drive device 500 is in contact with the outer circumference of the rotor 700 as the driven body. In the example shown in the drawing, the drive circuit 600 applies an AC voltage or a pulsating voltage between the pair of second electrode layers 50a and 50d and the first electrode layer 30, and the piezoelectric body layers 40a and 40d expand and contract in the direction of the arrow x in FIG. 22. In response to this, the vibrating body portion 512 of the piezoelectric drive device 500 is bent in the plane of the vibrating body portion 512 and is deformed into a meandering shape (S-shape), and the tip of the protrusion portion 520 performs reciprocating motion in the direction of the arrow y or performs elliptical motion. As a result, the rotor 700 rotates in a given direction z (a clockwise direction in FIG. 22) around the center 702 thereof. The three connecting portions 514 of the vibrating plate 510 are each provided at a position of a vibration knot (joint) of such a vibrating body portion 512.

Incidentally, in the case where the drive circuit 600 applies an AC voltage or a pulsating voltage between the other pair of second electrode layers 50b and 50c and the first electrode layer 30, the rotor 700 rotates in the opposite direction. Further, when the same voltage as applied to the pair of second electrode layers 50a and 50d (or the other pair of second electrode layers 50b and 50c) is applied to the second electrode layer 50e in the center, the piezoelectric drive device 500 expands and contracts in the longitudinal direction, and therefore, a force to be applied to the rotor 700 from the protrusion portion 520 can be further increased.

### 6. Device Using Piezoelectric Drive Device

The above-mentioned piezoelectric drive device 500 can apply a large force to a driven body by utilizing resonance, and can be applied to various devices. For example, the piezoelectric drive device 500 can be used as a drive device in various apparatuses such as a robot (also including an electronic component conveying device (IC handler)), a dosing pump, a timepiece calendar feeding device, and a printing device (for example, a sheet feeding mechanism, however, not applicable to a head since the vibrating plate is not caused to resonate in the piezoelectric drive device used for the head). Hereinafter, a representative embodiment will be described.

### 6.1. Robot

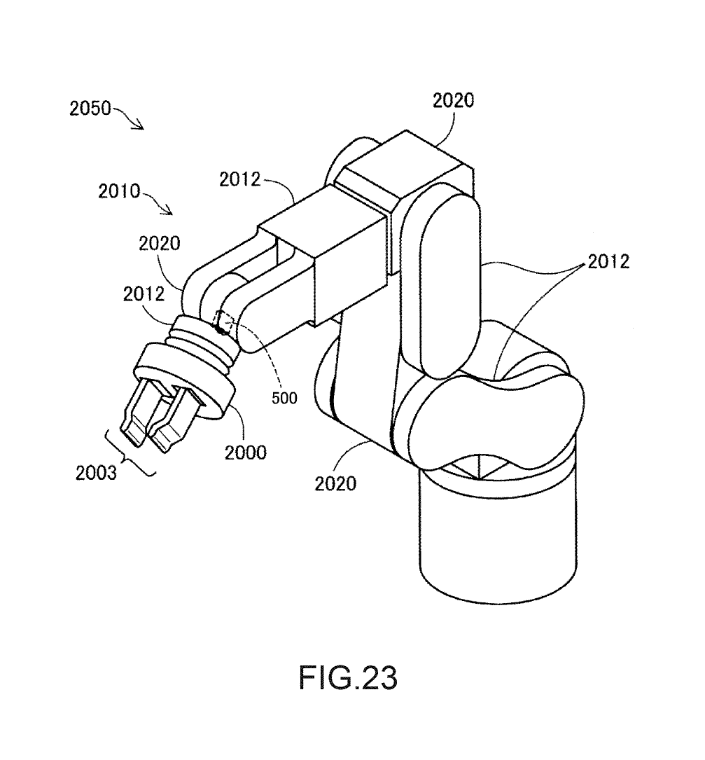

FIG. 23 is a view for illustrating a robot 2050 using the above-mentioned piezoelectric drive device 500. The robot 2050 has an arm 2010 (also referred to as “arm portion”) which includes a plurality of link portions 2012 (also referred to as “link members”) and a plurality of joint portions 2020 for connecting the link portions 2012 to each other in a rotatable or bendable state.

In each of the joint portions 2020, the above-mentioned piezoelectric drive device 500 is incorporated, and the joint portions 2020 can be rotated or bent at a given angle using the piezoelectric drive device 500. To the tip of the arm 2010, a robot hand 2000 is connected. The robot hand 2000 includes a pair of gripping portions 2003. Also in the robot hand 2000, the piezoelectric drive device 500 is incorporated, and it is possible to grip an object by opening and closing the gripping portions 2003 using the piezoelectric drive device 500. Further, the piezoelectric drive device 500 is also provided between the robot hand 2000 and the arm 2010, and it is also possible to rotate the robot hand 2000 with respect to the arm 2010 using the piezoelectric drive device 500.

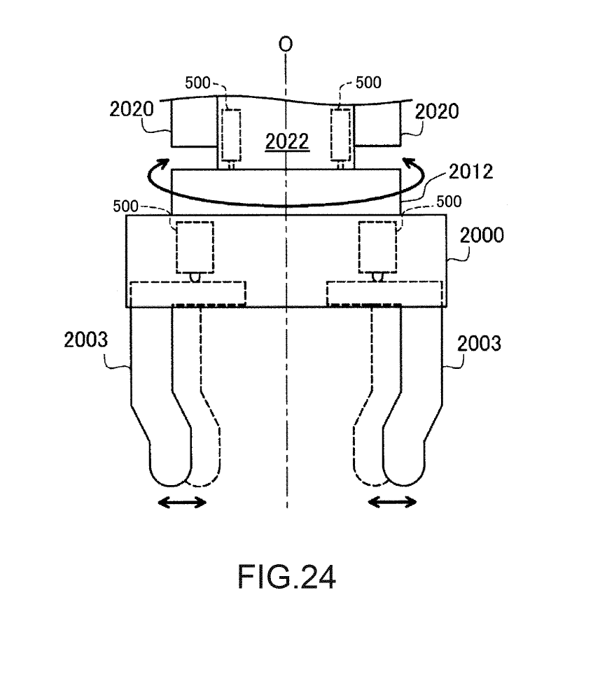

FIG. 24 is a view for illustrating a wrist portion of the robot 2050 shown in FIG. 23. The joint portions 2020 of the wrist interpose a wrist rotating portion 2022, and the link portion 2012 of the wrist is attached to the wrist rotating portion 2022 rotatably around the central axis O of the wrist rotating portion 2022. The wrist rotating portion 2022 includes the piezoelectric drive device 500, so that the piezoelectric drive device 500 rotates the link portion 2012 of the wrist and the robot hand 2000 around the central axis O. The plurality of gripping portions 2003 are provided upright on the robot hand 2000. The proximal end portion of the gripping portion 2003 can move in the robot hand 2000, and the piezoelectric drive device 500 is mounted on the base portion of the gripping portions 2003. According to this, by operating the piezoelectric drive device 500, the gripping portions 2003 can be moved to grip a target object. Incidentally, the robot is not limited to a single arm robot, and the piezoelectric drive device 500 can also be applied to a multi-arm robot in which the number of arms is two or more.

Here, in the inside of the joint portion 2020 of the wrist or the robot hand 2000, in addition to the piezoelectric drive device 500, an electric power line for supplying electric power to various devices such as a force sensor or a gyro sensor, a signal line for transmitting a signal, or the like is included, and thus a large number of wirings are necessary. Therefore, it was very difficult to dispose wirings inside the joint portion 2020 or the robot hand 2000. However, in the piezoelectric drive device 500 of the embodiment described above, a drive current can be made smaller than that of a general electric motor or a piezoelectric drive device in the related art, and therefore, wirings can be disposed even in a small space such as the joint portion 2020 (particularly a joint portion at the tip of the arm 2010) or the robot hand 2000.

### 6.2. Pump

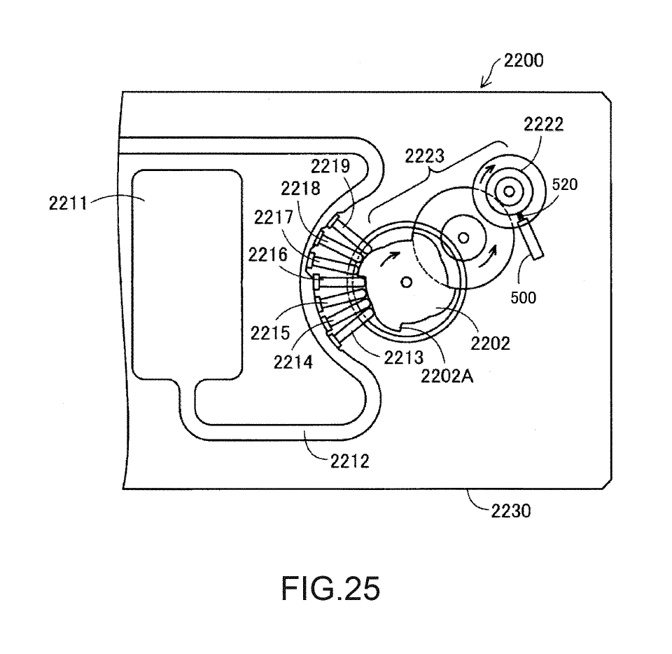

FIG. 25 is an explanatory view showing one example of a liquid feed pump 2200 utilizing the above-mentioned piezoelectric drive device 500. The liquid feed pump 2200 includes, in a case 2230, a reservoir 2211, a tube 2212, the piezoelectric drive device 500, a rotor 2222, a deceleration transmission mechanism 2223, a cam 2202, and a plurality of fingers 2213, 2214, 2215, 2216, 2217, 2218, and 2219.

The reservoir 2211 is a storage portion for storing a liquid to be transported. The tube 2212 is a tube for transporting the liquid to be sent from the reservoir 2211. The protrusion portion 520 of the piezoelectric drive device 500 is provided in a state of being pressed against the side surface of the rotor 2222, and the piezoelectric drive device 500 rotationally drives the rotor 2222. The rotational force of the rotor 2222 is transmitted to the cam 2202 through the deceleration transmission mechanism 2223. The fingers 2213 to 2219 are members for blocking the tube 2212. When the cam 2202 rotates, the fingers 2213 to 2219 are sequentially pressed outward in the radial direction by a projection portion 2202A of the cam 2202. The fingers 2213 to 2219 sequentially block the tube 2212 from the upstream side (the reservoir 2211 side) in the transportation direction. Due to this, the liquid in the tube 2212 is sequentially transported to the downstream side. By doing this, it is possible to realize the liquid feed pump 2200 capable of accurately feeding an extremely small amount of a liquid and also having a small size.

The arrangement of each member is not limited to one shown in the drawing. Further, a configuration in which a member such as a finger is not included and a ball or the like provided on the rotor 2222 blocks the tube 2212 may be adopted. The liquid feed pump 2200 as described above can be used for a dosing device or the like which administers a medicinal solution such as insulin to the human body. Here, by using the piezoelectric drive device 500 of the embodiment described above, a drive current becomes smaller than that of a piezoelectric drive device in the related art, and therefore, power consumption of the dosing device can be suppressed. Therefore, in the case where the dosing device is driven with a battery, the use of the piezoelectric drive device 500 is particularly effective.

The above-mentioned embodiments and variations are examples, and the invention is not limited thereto. For example, the respective embodiments and the respective variations can also be appropriately combined.

The invention includes substantially the same configurations (for example, configurations having the same functions, methods and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

The entire disclosures of Japanese Patent Application Nos. 2015-052219, filed Mar. 16, 2015, No. 2015-052220, filed Mar. 16, 2015, and No. 2015-052221 filed Mar. 16, 2015 are expressly incorporated by reference herein.

1. A method for producing a piezoelectric element, comprising:
a step of forming a first electrode layer;
a step of forming a piezoelectric body layer on the first electrode layer;
a step of forming a second electrode layer on the piezoelectric body layer;
a step of patterning the second electrode layer;
a step of patterning the piezoelectric body layer by wet etching; and
a step of forming an organic insulating layer on a side surface of the patterned piezoelectric body layer.

2. The method for producing a piezoelectric element according to claim 1, wherein the piezoelectric body layer is formed by repeating formation of a precursor layer by a liquid-phase method and crystallization of the precursor layer.

3. The method for producing a piezoelectric element according to claim 1, wherein the material of the organic insulating layer is a photosensitive material.

4. The method for producing a piezoelectric element according to claim 3, wherein the Young's modulus of the organic insulating layer is 1 GPa or more.

5. The method for producing a piezoelectric element according to claim 1, wherein the thickness of the organic insulating layer is 1.5 times or more and 3 times or less the thickness of the piezoelectric body layer.

6. The method for producing a piezoelectric element according to claim 1, wherein the thickness of the piezoelectric body layer is 1 μm or more and 10 μm or less.

7. A piezoelectric element, comprising:
a first electrode layer;
a piezoelectric body layer provided on the first electrode layer;
a second electrode layer provided on the piezoelectric body layer; and
an organic insulating layer provided on a side surface of the piezoelectric body layer, wherein
the piezoelectric body layer is formed by repeating formation of a precursor layer by a liquid-phase method and crystallization of the precursor layer to form a stacked body, and patterning the stacked body by wet etching.

8. A piezoelectric drive device, comprising:
a vibrating plate; and
the piezoelectric element according to claim 7 provided on a surface of the vibrating plate.

9. A robot, comprising:
a plurality of link portions;
a joint portion for connecting the plurality of link portions; and
the piezoelectric drive device according to claim 8 which rotates the plurality of link portions at the joint portion.

10. A pump, comprising:
the piezoelectric drive device according to claim 8;
a tube for transporting a liquid; and
a plurality of fingers for blocking the tube by driving the piezoelectric drive device.

11. A method for producing a piezoelectric element, comprising:
a step of forming a first electrode layer;
a step of forming a piezoelectric body layer on the first electrode layer;
a step of forming a second electrode layer on the piezoelectric body layer;
a step of forming a resist layer on the second electrode layer;
a step of patterning the second electrode layer by wet etching;
a step of patterning the piezoelectric body layer by wet etching; and
a step of removing an eaves portion of the second electrode layer generated by side etching in the step of patterning the piezoelectric body layer by wet etching.

12. The method for producing a piezoelectric element according to claim 11, wherein
the step of forming the second electrode layer includes
a step of forming an adhesion layer, and
a step of forming an electrically conductive layer on the adhesion layer, and
in the step of removing the eaves portion,
after the adhesion layer is removed, the electrically conductive layer is removed.

13. The method for producing a piezoelectric element according to claim 11, wherein the second electrode layer contains at least one of copper and gold.

14. The method for producing a piezoelectric element according to claim 11, wherein the thickness of the second electrode layer is 50 nm or more and 10 μm or less.

15. The method for producing a piezoelectric element according to claim 11, wherein the thickness of the piezoelectric body layer is 1 μm or more and 10 μm or less.

16. A piezoelectric element for an ultrasonic motor, comprising:
a first electrode layer;
a piezoelectric body layer provided on the first electrode layer; and
a second electrode layer provided on the piezoelectric body layer, wherein
the second electrode layer contains copper, and
the thickness of the second electrode layer is 50 nm or more and 10 μm or less.

17. The piezoelectric element for an ultrasonic motor according to claim 16, wherein the second electrode layer includes
an adhesion layer,
an electrically conductive layer provided on the adhesion layer and containing the copper, and
an antioxidation layer provided on the electrically conductive layer.

18. The piezoelectric element for an ultrasonic motor according to claim 16, wherein the material of the antioxidation layer is the same as the material of the adhesion layer.

19. The piezoelectric element for an ultrasonic motor according to claim 16, wherein the material of the antioxidation layer is a polymer.

20. A method for producing a piezoelectric element for an ultrasonic motor, comprising:
a step of forming a first electrode layer;
a step of forming a piezoelectric body layer on the first electrode layer; and
a step of forming a second electrode layer on the piezoelectric body layer, wherein
the second electrode layer contains copper, and
the thickness of the second electrode layer is 50 nm or more and 10 μm or less.

21. (canceled)

22. (canceled)

23. (canceled)

24. (canceled)

25. (canceled)

26. (canceled)
